(19) United States
(12) Patent Application Publication
(10) Pub. No.: US 2023/0000670 A1
(43) Pub. Date: Jan. 5, 2023

(54) DETACHABLE COOLING APPARATUS, ASSOCIATED SYSTEM, AND METHOD OF DEPLOYMENT

(71) Applicant: Icahn School of Medicine at Mount Sinai, New York, NY (US)

(72) Inventors: Turner S. BAKER, Brooklyn, NY (US); Anthony COSTA, Upper Nyack, NY (US); Peter BACKERIS, Staten Island, NY (US); Christopher P. KELLNER, New York, NY (US); Hazem SHOIRAH, New York, NY (US)

(21) Appl. No.: 17/778,289

(22) PCT Filed: Nov. 20, 2020
(86) PCT No.: PCT/US2020/061541

§ 371 Date: May 19, 2022

Related U.S. Application Data
(60) Provisional application No. 62/938,916, filed on Nov. 21, 2019.

(51) Int. Cl. A61F 7/12; A61F 7/00; A61M 25/00

(52) U.S. Cl. CPC A61F 7/12; A61F 7/0085; A61M 25/0054; A61F 2007/0096; A61F 2007/0086; A61F 2007/126; A61F 2007/0056; A61M 2025/0059

(57) ABSTRACT

A detachable cooling apparatus comprises: a distal miming catheter forming a distal lumen that provides liquid as an input and a proximal miming catheter forming a proximal lumen that receives the liquid as an output, where the proximal miming catheter is connected to the distal running catheter. A focal hypothermia-inducing fluidics system comprises a thermal management and flow system (TMFS) that is operable to alter a liquid to a specific temperature and to regulate a flow rate, a closed-circuit flow system with a detachable cooling apparatus, a distal sensor array, a pump for moving the liquid through the TMFS, an inflow port that receives the liquid from the proximal running catheter, a plurality of capillary tubes that cool the liquid, and an outflow port that returns the liquid to the distal miming catheter.

### CROSS-REFERENCE TO RELATED APPLICATION

The present application claims the benefit of and priority to U.S. provisional patent application Ser. No. 62/938,916, filed Nov. 21, 2019, which is hereby expressly incorporated by reference in its entirety.

### TECHNICAL FIELD

The present specification relates generally to instruments and methods to treat intracerebral hemorrhage (ICH) and more particularly, to a detachable cooling apparatus and associated system to be deployed during brain surgery for treating symptoms associated with intracerebral hemorrhage (ICH), craniectomy-requiring surgeries, or intraparenchymal operations.

### BACKGROUND

Bleeding inside the brain is known as intracerebral hemorrhage (ICH). ICH can occur as a result of traumatic brain injury or can occur spontaneously. Common etiologies for atraumatic ICH include hypertensive arteriopathy, cerebral amyloid angiopathy, hemorrhagic transformation of ischemic stroke, an underlying vascular lesion, hemorrhagic tumor, or other less common causes.

Over five million hemorrhagic strokes occur every year worldwide. There are approximately 70,000 cases of ICH in the United States per year. ICH is the deadliest form of stroke with approximately 40% mortality within 30 days of the primary insult. Six months after the bleed only 20% of patients regain functional independence in their daily activities.

Mechanical stress and destruction of cerebral tissue due to hematoma formation rapidly follows the hemorrhagic event and is associated with swelling of brain tissue surrounding the site of injury. Neurotoxic components of the hematoma induce a secondary, subacute injury manifested as perihematomal edema (PHE). Formation of PHE begins within the first four hours and accelerates rapidly, reaching 60% of peak absolute volume 24 hours after the hemorrhage. Although the most rapid expansion occurs in the first 48 hours, the edema continues to increase until an average of 12 days after the hemorrhage. The growth of the edema correlates with a decline in neurological function. No treatment has yet been discovered to mitigate PHE or improve outcome after ICH. Current medical management focuses on limiting the primary injury by controlling bleeding, decreasing the chance of rebleeding, and providing supportive care for the patients. The Surgical Treatment in Intracerebral Hemorrhage (STICH) and the Surgical Treatment in Lobar Intracerebral Hemorrhage (STICH II) trials both failed to demonstrate an improvement in the primary outcome of improved neurologic function in the surgical group after ICH. Attempts at aggressive blood pressure control through the Intensive Blood Pressure Reduction in Acute Cerebral Hemorrhage (INTERACT II) and Antihypertensive of Acute Cerebral Hemorrhage (ATACH II) trails also missed their primary outcomes of improved functional status. In addition, the Recombinant Factor VIIa in Acute Intracerebral Hemorrhage (FAST) administering FACTOR VII acutely did not show a benefit while the Platelet transfusion versus standard care after acute stroke due to spontaneous cerebral hemorrhage associated with antiplatelet therapy (PATCH) trial addressing hemostasis did not show a functional improvement while the PATCH trial actually demonstrated that giving platelets to patients on antiplatelet agents was deleterious.

Studies have identified a therapeutic role of hypothermia as a tool to treat ICH. Hypothermia is one of the earliest and most thoroughly studied neuroprotectants that exerts its effects through multiple mechanisms. Acutely, hypothermia reduces metabolic rate and the release of excitatory neurotransmitters and improves glucose metabolism. Subacutely, it reduces cell-mediated inflammation with the reduction in inflammatory markers and down-regulation of pro-apoptotic BAX and up-regulation of anti-apoptotic BCL-2 gene expression.

Systemic hypothermia is the practice of inducing hypothermia by reducing the core temperature of the body achieving hypothermia of all organs including the target organ: the brain. The application of systemic hypothermia has been studied in multiple conditions that result in neurological injury; most notably post-cardiac arrest, neonatal hypoxia, and stroke. However, systemic hypothermia carries specific risks related to the non-selective cooling of the organ systems, which include bradyarrhythmias, coagulopathies, metabolic derangements, immune suppression, and shivering which may cause as much damage as the ICH itself.

### SUMMARY

Embodiments generally relate to a detachable cooling apparatus that comprises a distal running catheter forming a distal lumen that provides liquid as an input and a proximal running catheter forming a proximal lumen that receives the liquid as an output, wherein the proximal running catheter is connected to the distal running catheter. In some embodiments, the detachable cooling apparatus further includes a heat-exchanger region comprising a high conductivity material that cools surrounding tissue with the liquid. In some embodiments, the detachable cooling apparatus further includes a connector that allows for attachment, detachment, and alignment of the distal running catheter and the proximal running catheter to line up to one or more catheters associated with a fluidics pump and thermal-regulating mechanism. In some embodiments, the detachable cooling apparatus further includes a sensor array that is operable to determine at least one of a temperature of an internal liquid or surrounding tissue, a pressure of the internal liquid or surrounding tissue, a flow rate of the internal liquid or surrounding tissue, and biological properties of the internal liquid or surrounding tissue and an external controller operable to modify the at least one of the temperature, the pressure, and the flow rate. In some embodiments, the sensor array is located at a far distal end of the distal running catheter or at any section of the distal running catheter. In some embodiments, the detachable cooling apparatus further includes a heat-exchanger region comprising a high conductivity loop that cools surrounding tissue with the liquid. In some embodiments, the high conductivity loop is at a substantially 180-degree angle. In some embodiments, the detachable cooling apparatus further includes a heat-exchanger region comprising a high conductivity coiled proximal running catheter that wraps around a distal running catheter that connects the distal running catheter to the proximal running catheter and that cools surrounding tissue with the liquid. In some embodiments, the detachable cooling apparatus further includes a heat-exchanger region comprising a high conductivity inflatable member, such as a bag or balloon, that receives the liquid from the distal running catheter as the input to a distal most region of the inflatable member and a proximal lumen that receives the liquid from the inflatable member as the output at a proximal most point of the inflatable member that connects the distal running catheter to the proximal running catheter and that cools surrounding tissue with the liquid. In some embodiments, the detachable cooling apparatus further includes a fluidics midsection that thermally insulates the liquid that is input into the distal running catheter until it reaches the heat-exchanger region. In some embodiments, the fluidics midsection includes a polyurethane or silicone section that thermally insulates the liquid. More specifically, this midsection is designed to have the required flexibility while also remaining thermally insulating. For example, it is desirable for this flexible midsection (also described herein as being a flexible intermediate region) to be able to navigate through two 90 degree turns without kinking. As described herein, the expression “kinking” describes a situation in which at least one of the lumens (e.g., distal running lumen or proximal running lumen) is substantially occluded (i.e., greater than 40% occlusion of the at least one lumen due to bending/collapsing of the lumen wall). A silicone based midsection can deliver these properties/features. In some embodiments, the detachable cooling apparatus further includes an additional set of lumens and ports that provide at least one of a drug, a lavage, ventricular drain, or sensor wires. In some embodiments, the detachable cooling apparatus further includes a connection that attaches the proximal end of the detachable cooling apparatus to a fluidics cooling and pump system and at least one additional lumen that introduces the drug, lavage, drain or sensor through a distal port located anywhere between the connection and a distal-most end of the detachable cooling apparatus. In some embodiments, the connection is a screw lock. In some embodiments, connection is a luer lock. As described herein, the connection system that is associated with the detachable cooling apparatus must allow for tunneling under the skin as described herein. The present disclosure describes different embodiments in which connectors are configured to be attached to the catheter structure (the detachable cooling apparatus which itself can tunnel under the skin) after the catheter structure has passed through the surgical equipment and tunneled under the skin or alternatively, the connectors themselves are small enough to be tunneled under the skin. In addition, the catheter structure or connector needs to be able to attach to common metal stylets that are commonly used in surgery. Commonly, this can be accomplished with silicone fitting over a barbed-tube connector on the stylet.

In some embodiments a method comprises performing a surgical evacuation of an intracranial hematoma in a patient, installing an apparatus within a remaining hematoma cavity of the patient, using imaging to confirm placement of the apparatus in a brain of the patient, activating the apparatus to induce neuroprotection from within the remaining hematoma cavity, operating the apparatus until after an end of surgery, and removing the apparatus without an additional surgical operation. Suitable imaging equipment includes but is not limited to MRI, CT, and ultrasound. Alternatively, direct visualization can be used, such as using an endoscope (which can be associated with a SurgiScope as described herein).

In some embodiments, a focal hypothermia-inducing fluidics system comprises: a thermal management and flow system (TMFS) that is operable to alter a liquid to a specific temperature and to regulate a flow rate, the TMFS including a pump for moving the liquid through the TMFS, an inflow port that receives the liquid from a proximal running catheter, a cooling unit to cool the liquid, and an outflow port that returns the liquid to a distal running catheter, a closed-circuit flow system with a detachable cooling apparatus, and a distal sensor array that is operable to determine at least one of a temperature, a pressure, the flow rate, and a biological property of the liquid and a surrounding region. In some embodiments, the TMFS further includes a cooling unit that physically contacts a plurality of capillary tubes to increase or decrease the temperature of the liquid, wherein the cooling unit operates using at least one of peltier cooling, liquid cooling, evaporative cooling, and passive cooling. In some embodiments, the pump of the TMFS is at least one of a positive displacement pump, a rotary-type pump, a gear pump, a screw pump, a peristaltic pump, a rotary vane pump, a centrifugal pump, an impulse pump, a hydraulic ram pump, a pulser, an airlift pump, a velocity pump, a radial-flow pump, and a centrifugal and axial-flow pump. In some embodiments, the focal hypothermia-inducing fluidics system further includes a controller that is operable to communicate with a sensor array to regulate temperature and flow rate. In some embodiments, the detachable cooling apparatus includes a heat-exchanger region, and the focal hypothermia-inducing fluidics system further includes a fluidics midsection that thermally insulates the liquid that is input into the distal running catheter until it reaches the heat-exchanger region. In some embodiments, the fluidics midsection includes a polyurethane section that thermally insulates the liquid. In some embodiments, the focal hypothermia-inducing fluidics system further includes a set of lumens and ports that provide at least one of a drug, a lavage, ventricular drain, or sensor wires. In some embodiments, the detachable cooling apparatus includes a distal running catheter and the distal sensor array is located at a far distal end of the distal running catheter or a midsection of the distal running catheter. In some embodiments, the focal hypothermia-inducing fluidics system further includes a drug infusion port. In some embodiments, focal hypothermia-inducing fluidics system further includes a user interface that is operable to provide a user with an option for changing the temperature or flow rate of the liquid in the system.

In some embodiments, a method for deployment of a detachable cooling apparatus intracranially uses the focal hypothermia inducing fluidics system of claim 19 following intracranial hemorrhage evacuation. In some embodiments, a method for deployment of a detachable cooling apparatus intracranially uses the focal hypothermia inducing fluidics system by following at least one of intracerebral hemorrhage (ICH) evacuation, craniectomy, and intraparenchymal operations. In some embodiments, wherein a proximal section of the detachable cooling apparatus remains external to a cranium of a patient and is tunneled beneath skin of the patient. In some embodiments, the method further includes removing the detachable cooling apparatus by pulling a proximal end of the detachable cooling apparatus outward from a skull of a patient. In some embodiments, a method for deploying the detachable cooling apparatus is deployed intracranially following an intraparenchymal operation.

### BRIEF DESCRIPTION OF THE DRAWING FIGURES

The disclosure is illustrated by way of example, and not by way of limitation, in the accompanying drawings in which like reference numerals are used to refer to similar elements.

FIG. 1 is a block diagram of an exemplary focal hypothermia-inducing fluidics system to treat intracerebral hemorrhages according to some embodiments;

FIG. 2 is a block diagram of another exemplary focal hypothermia-inducing fluidics system that is modified to include a drug delivery system;

FIG. 3A is a side view of a distal end region of a loop embodiment of the detachable cooling apparatus according to one exemplary embodiment;

FIG. 3B is a cross-sectional top view of the detachable cooling apparatus of FIG. 3A;

FIG. 3C is a cross-sectional view taken along the line A-A of FIG. 3B;

FIG. 3D is another cross-sectional view of the detachable cooling apparatus rotated 90 degrees relative to FIG. 3B;

FIG. 3E is a cross-sectional view taken along the line B-B of FIG. 3D;

FIG. 3F is side perspective view of the detachable cooling apparatus;

FIG. 3G is a side perspective view of the detachable cooling apparatus of FIG. 3A;

FIG. 4A is a side view of a distal end region of a loop embodiment of the detachable cooling apparatus according to another exemplary embodiment;

FIG. 4B is a cross-sectional top view of the detachable cooling apparatus of FIG. 4A;

FIG. 4C is a cross-sectional view taken along the line A-A of FIG. 4B;

FIG. 4D is another cross-sectional view of the detachable cooling apparatus rotated 90 degrees relative to FIG. 4B;

FIG. 4E is a cross-sectional view taken along the line B-B of FIG. 4D;

FIG. 4F is side perspective view of the detachable cooling apparatus;

FIG. 4G is a side perspective view of the detachable cooling apparatus of FIG. 4A showing transparent and opaque portions;

FIG. 5A is a side view of a distal end region of a spiral embodiment of the detachable cooling apparatus according to one exemplary embodiment;

FIG. 5B is a cross-sectional top view of the detachable cooling apparatus of FIG. 5A;

FIG. 5C is a cross-sectional view taken along the line A-A of FIG. 5B;

FIG. 5D is another cross-sectional view of the detachable cooling apparatus rotated relative to FIG. 5B;

FIG. 5E is a cross-sectional view taken along the line B-B of FIG. 5D;

FIG. 5F is side perspective view of the detachable cooling apparatus;

FIG. 5G is a side perspective view of the detachable cooling apparatus showing of FIG. 5A transparent and opaque portions;

FIGS. 6A-6E are cross-sectional views a primary fluidics cross-section and alternative embodiments;

FIG. 7A is a side perspective view of an exemplary connection between a detachable cooling apparatus and a thermal management and flow system;

FIG. 7B is a side elevation thereof;

FIG. 7C is a cross-sectional view thereof;

FIG. 8A is a top plan view of another detachable cooling apparatus;

FIG. 8B is a cross-sectional view thereof;

FIG. 9 is a block diagram of a thermal management and flow system according to one exemplary embodiment;

FIG. 10 illustrates an exemplary user interface for managing the detachable cooling apparatus according;

FIGS. 11A-11H illustrate the brain during deployment, operation, and removal of the detachable cooling system according to one embodiment;

FIG. 12 is a flow diagram of a method for installing and removing an apparatus according to some embodiments;

FIGS. 13A-13F shows a series of steps illustrating the positioning of the detachable cooling apparatus through a burr hole into the brain;

FIGS. 14A and 14B show attachment of a metal stylet to the detachable cooling apparatus;

FIG. 15 is a side view schematically showing various regions of one detachable cooling apparatus;

FIGS. 16A-16D illustrate various view of a detachable cooling apparatus according to one embodiment;

FIG. 17 is a side view of a detachable cooling apparatus according to one embodiment; and

FIGS. 18A and 18B illustrate a detachable cooling apparatus with an inflatable member in a deflated (collapsed) state in FIG. 18A and an inflated state in FIG. 18B.

### DETAILED DESCRIPTION OF CERTAIN EMBODIMENTS

The present specification generally discloses a fluid-based, closed-circuit cooling system that is used to treat symptoms associated with intracerebral hemorrhage (ICH), craniectomy-requiring surgeries, or intraparenchymal operations by placing a detachable cooling apparatus in the evacuation cavity after intracerebral hemorrhage evacuation, attaching it to a fluidics cooling system and inducing targeted temperature management. The detachable cooling apparatus is placed and connected to the fluidics cooling system during surgery, can remain operative for weeks, and can be removed without the need for additional operations. The detachable nature of the cooling apparatus allows for easy placement of the apparatus during surgery and increases the ability to tunnel the device beneath the skin. The detachable cooling apparatus may be removed by pulling the proximal end of the detachable cooling apparatus outward from the skull of the patient. This approach is different from current and traditional therapeutic hypothermia strategies that focus on systemic cooling by limiting the area of temperature modulation to the area of injury and the immediate vicinity, while also operating within the standard procedures currently employed to surgically treat ICH. The detachable cooling apparatus can help maximize the neuroprotective properties of hypothermia via temperature reduction in the perihematomal region and can help reduce or limit the complications associated with systemic hypothermia include shivering, coagulopathy, and infection. Definitions of terms will be as such: “Cooling” or any variation thereof will be defined as any reduction in temperature relative to the primary area of insult within the patient's brain. “Loop” will be defined as any shape wherein the starting point and ending point of a fluidics system are immediately adjacent. “Bag” will be defined as any thin-membraned, volume filling compartment that conforms to the surrounding area. “High conductivity material” is defined as any material with low thermal resistance, specifically, plastics with thermal conductivities of around or greater than 0.5 Watts/meter-° Kelvin as one exemplary property.

### Exemplary System

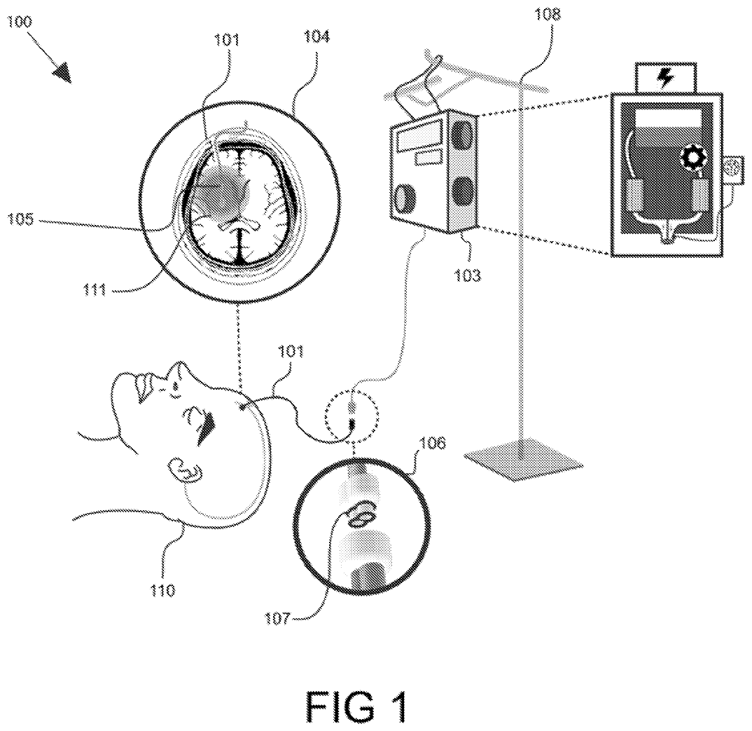

FIG. 1 illustrates a block diagram of an exemplary neural hypothermia-inducing fluidics system 100 to treat symptoms associated with intracerebral hemorrhage (ICH), craniectomy-requiring surgeries, or intraparenchymal operations by placing a detachable cooling apparatus (device) 101 in the evacuation cavity after intracerebral hemorrhage evacuation and inducing targeted temperature management as described herein.

The hypothermia-inducing fluidics system 100 preferably includes a closed-circuit flow system with the detachable cooling apparatus 101 and a thermal management and flow system 103 for monitoring and/or controlling the operation of the detachable cooling apparatus 101. The detachable cooling apparatus 101 is placed in an evacuation cavity of a patient 110. The system 100 is configured such that a cooled liquid is delivered to the target area for inducing the targeted temperature management and more particularly, the use of the cooled liquid and design of the system maximizes neuroprotective properties of hypothermia via temperature reduction in the perihematomal region. In one embodiment, the cooled liquid comprises a saline solution; however, other suitable solutions can be used.

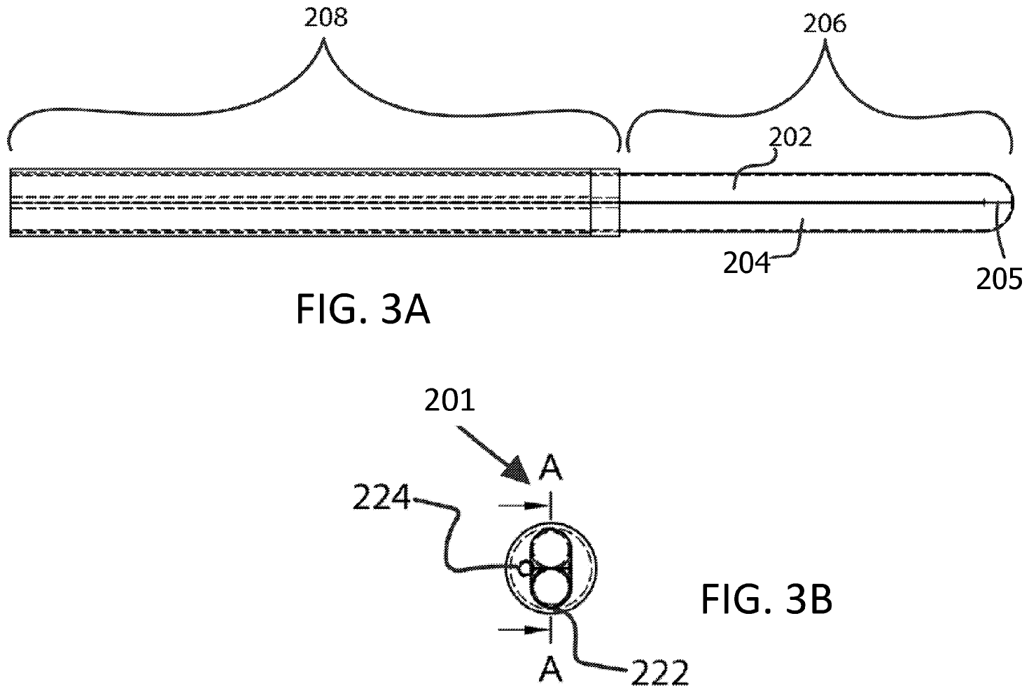

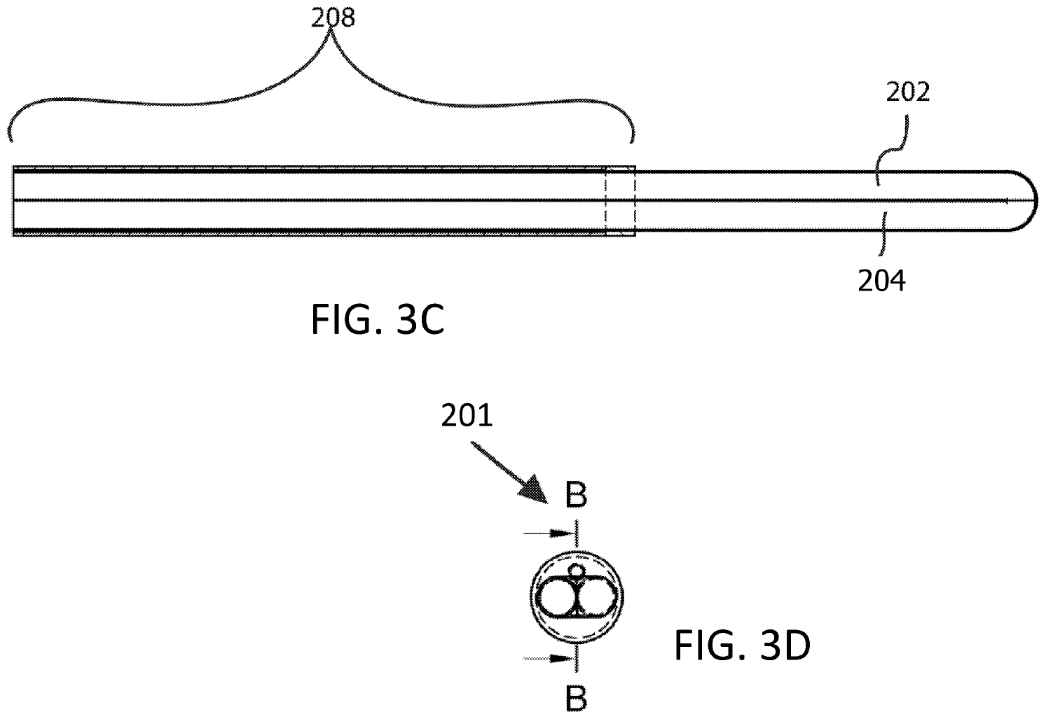

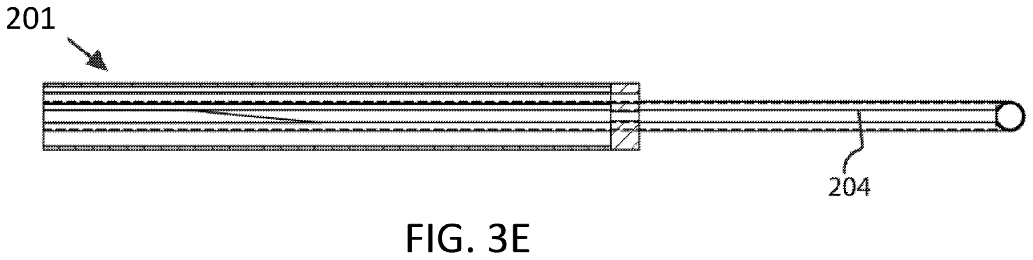

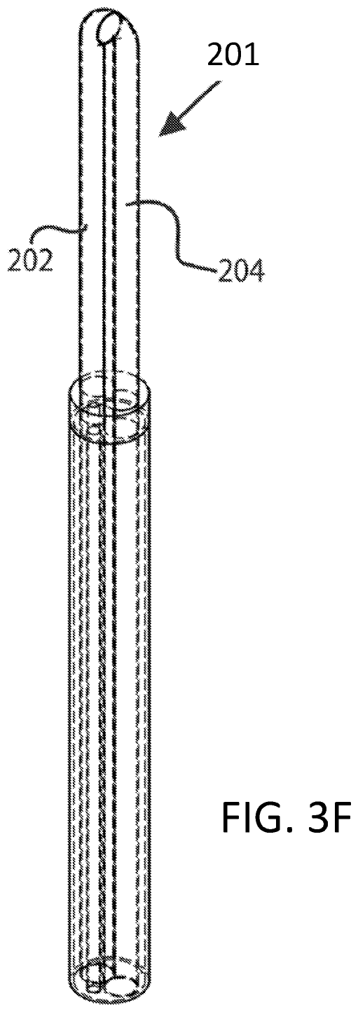

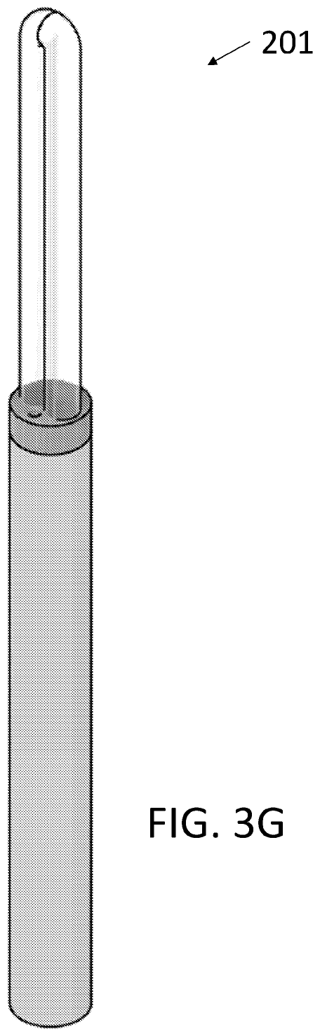

In some embodiments, the detachable cooling apparatus 101 includes a distal running catheter and a proximal running catheter (See, e.g., FIGS. 3A-3G), both comprising a thermally insulated midsection that is designed to insulate cooled liquid received from an external system until it reaches a heat-exchanger region 105 of the detachable cooling apparatus 101. Both the distal and proximal running catheters define the heat-exchanger region 105. As is known, a heat exchanger is a system that is used to transfer heat between two or more fluids (e.g., the cooled liquid and the tissue at the target site). In the present system 100, the heat exchanger region 105 is designed such that the cold temperature of the cooled liquid is transferred to the target site (e.g., tissue) within the patient's brain. It will be appreciated that the detachable cooling apparatus 101 is thus defined by a catheter body that can be made up of the distal running catheter and the proximal running catheter. The distal running catheter and the proximal running catheter can be two separate structures or can be a single integral structure that includes two discrete lumens (i.e., distal running and proximal running) that are separated from one another. In general, the detachable cooling apparatus 101 can be referred to as being a catheter structure.

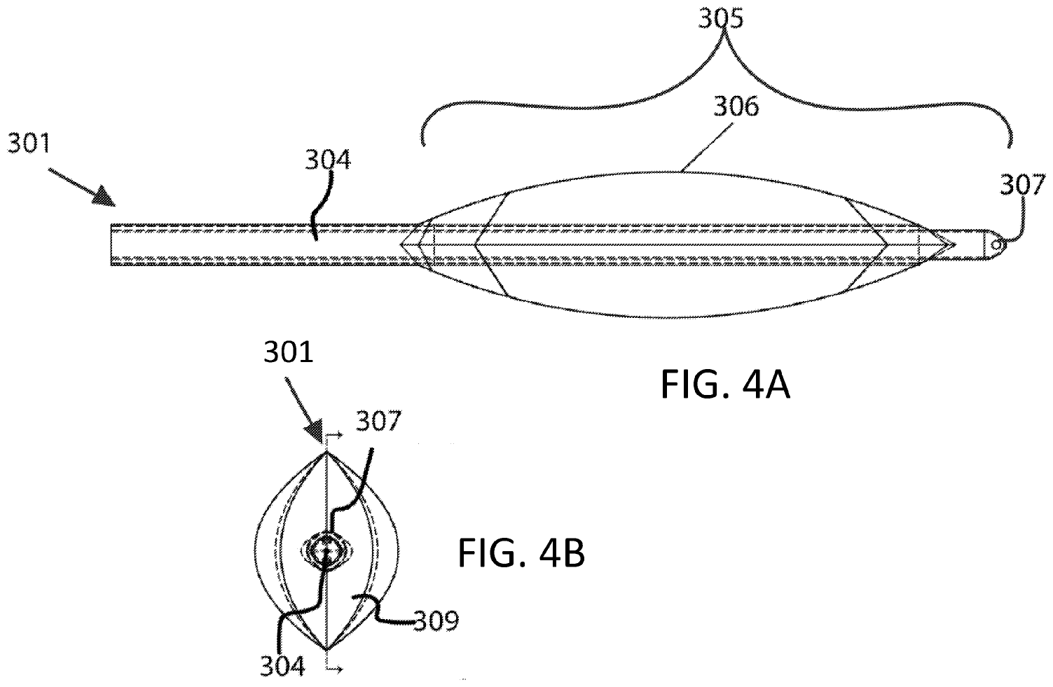

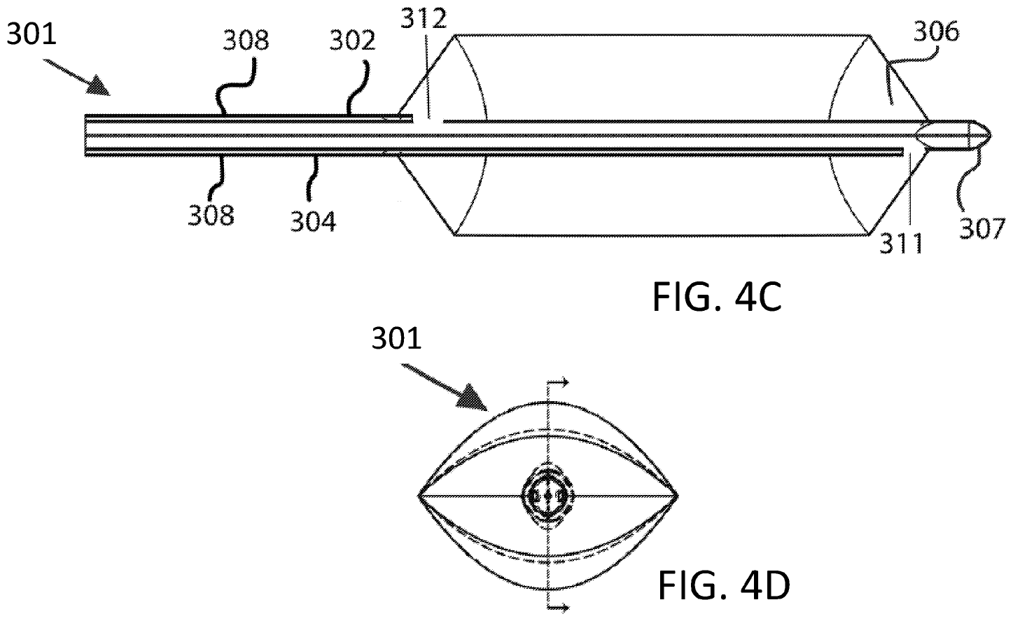

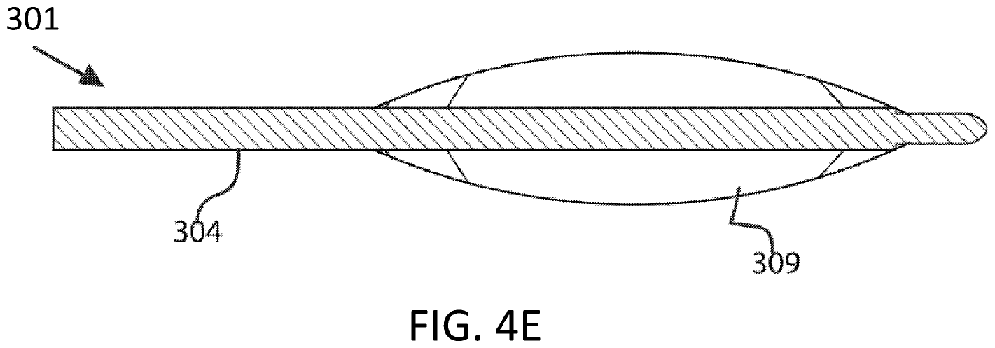

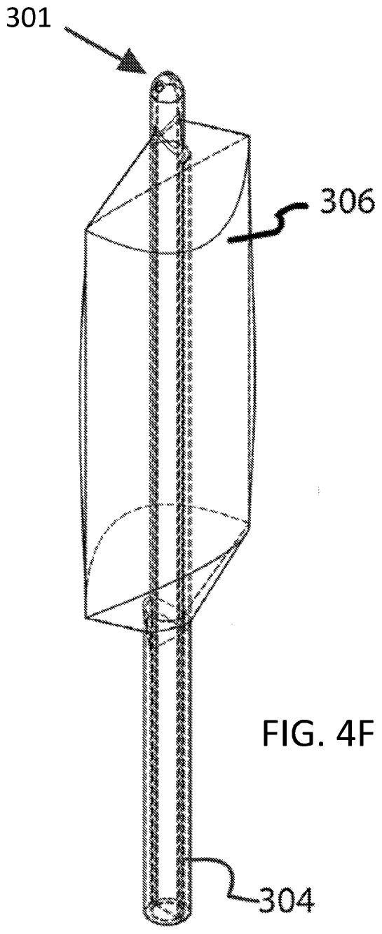

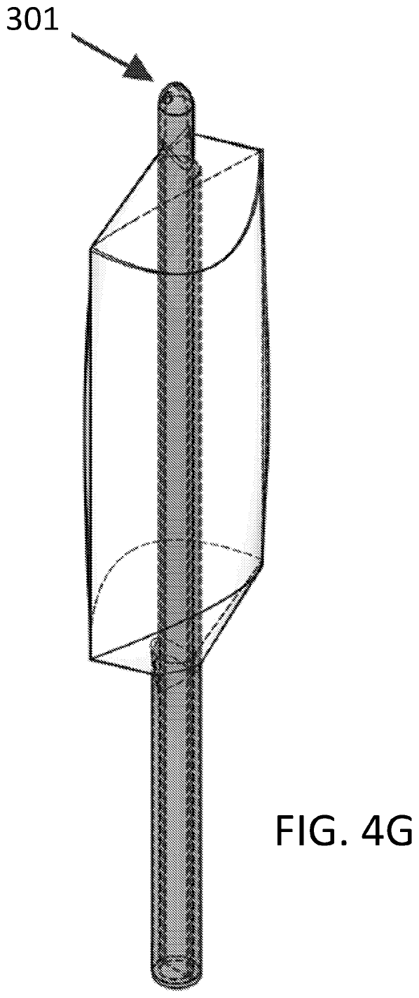

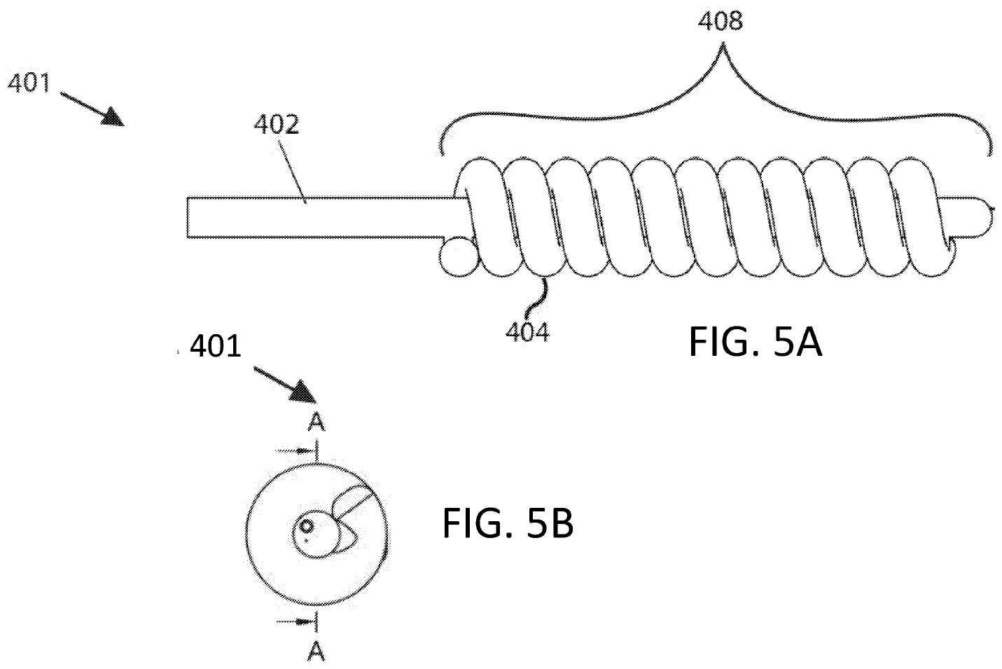

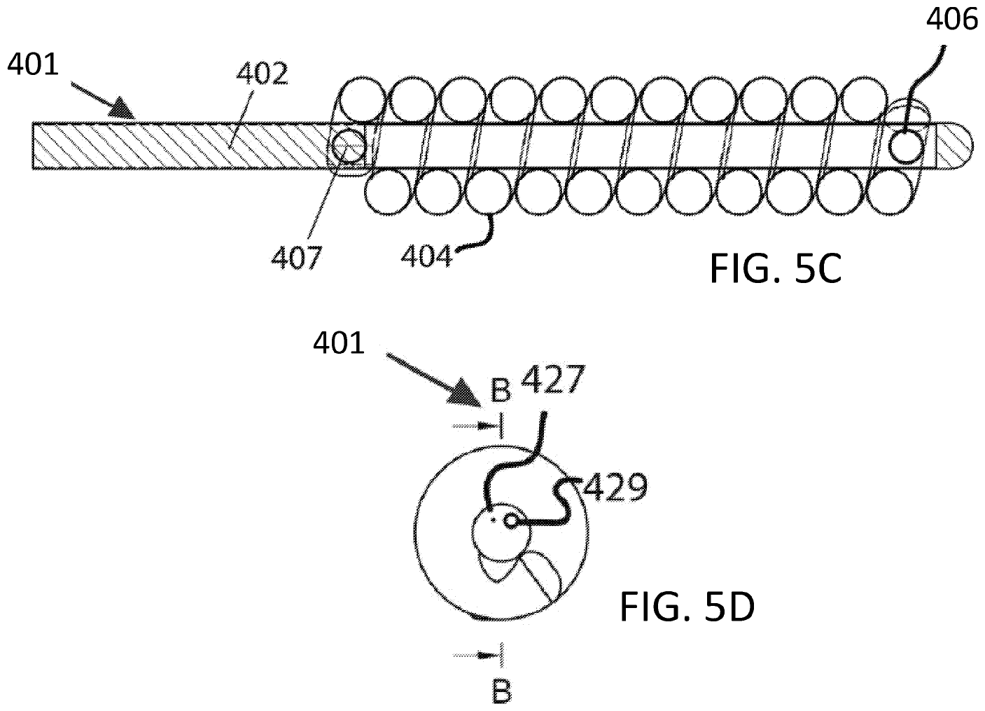

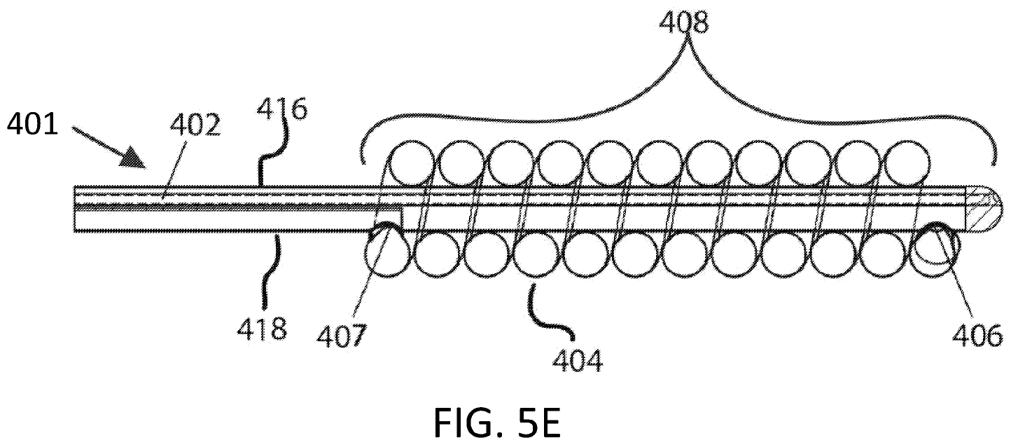

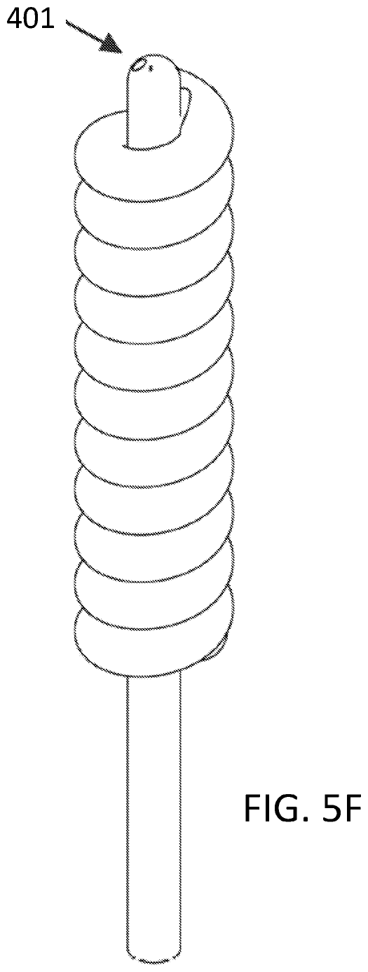

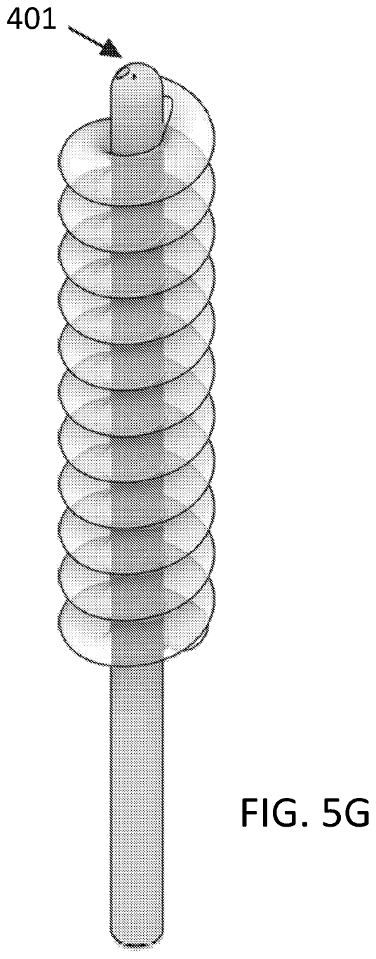

FIG. 1 includes a cross-section 104 of the patient's brain, which illustrates the detachable cooling apparatus 101 as entering the brain and including a temperature-controlled radius 111 around the heat exchanger region 105 of the detachable cooling apparatus 101 contained within the brain. In some embodiments, the proximal end of the detachable cooling apparatus 101 is securely attached to the patient's 110 skull. In some embodiments, the proximal section of the detachable cooling apparatus 101 may remain external to the cranium and is able to be tunneled beneath the skin of the patient. The system 100 may include a closed loop of the cooled liquid as described below with reference to FIGS. 3A-3G, a bag (inflatable member) of cooled liquid as described below with reference to FIGS. 4A-4G, or a spiral of cooled liquid as described below with reference to FIGS. 5A-5G. All of these embodiments are closed-circuit systems in which cooled liquid is delivered to a heat transfer location (heat-exchanger location) at which heat transfer occurs and then the liquid, in a warmer condition, is returned to be recooled and then the process continues.

FIG. 1 shows an enlarged section 106 of a connection between the detachable cooling apparatus 101 and the fluidics system which is designed to deliver the cooled liquid. In some embodiments, the connection may be a male-female luer-lock; however, any number of other types of connections can be used to provide a secure, fluid sealed connection between these two parts. Both the detachable cooling apparatus 101 and the fluidics midsection include two catheters 107: a distal running catheter that provides the cooled liquid to the evacuation cavity of the patient 110 and a proximal running catheter that returns liquid to the thermal management and flow system 103. The connected, these two catheters of the detachable cooling apparatus 101 and the fluidics midsection are in sealed, fluid communication to allow flow of the cooled liquid to the heat exchanger region 105 and also the return flow of the liquid after it has undergone heat transfer in the heat exchanger region 105 as described below.

The temperature of the cooled liquid is altered to a precise (inputted) value (within acceptable tolerances) within the thermal management and flow system 103 and is pumped down a distal running catheter (distal running lumen) through the detachable connection 107 and into the detachable cooling apparatus 101. The distal running catheter of the detachable cooling apparatus 101 provides the cooled liquid to the heat-exchanger region 105. The cooled liquid absorbs heat from the surrounding tissue through the heat-exchanger region 105. The liquid is then transmitted to a proximal running catheter and flows back to the system 103 via the proximal running catheter (proximal running lumen) of the detachable cooling apparatus 101.

The proximal running catheter (proximal running lumen) thus returns the liquid to the thermal management and flow system 103. In some embodiments, the thermal management and flow system 103 includes a pump for moving the liquid through the thermal management and flow system 103 or a liquid reservoir to de-air or promote rapid re-cooling of the cooling liquid. In some embodiments, the thermal management and flow system 103 includes an inflow port that receives the heated liquid from the proximal running catheter as it is returned from the heat-exchanger region 105, a plurality of capillary tubes that cool the liquid, and an outflow port that returns temperature-specific cooled liquid to the distal running catheter of the fluidics midsection. In some embodiments, the thermal management and flow system 103 includes an attachment, in this case a loop made from a sturdy material such as metal or plastic, for hanging the thermal management and flow system 103 to an IV rack 108 or similar upstanding structure. Alternatively, the thermal management and flow system 103 can be part of a standalone unit (console) that can includes wheels to allow the unit to move moved from one location to another.

In some embodiments, the thermal management and flow system 103 includes a cooling unit that physically contacts the plurality of capillary tubes to increase or decrease the temperature of the liquid, wherein the cooling unit may operate using at least one of peltier cooling, liquid cooling, evaporative cooling, and passive cooling.

In some embodiments, the thermal management and flow system includes a pump that induces fluidics flow. This pump may operate as a positive displacement pump (including rotary-type, gear, screw, peristaltic, and rotary vane), a centrifugal pump, impulse pump (including hydraulic ram, pulser, and airlift), or a velocity pump (including radial-flow/centrifugal and axial-flow). The pumping action thus causes the liquid to flow in the closed-circuit flow path.

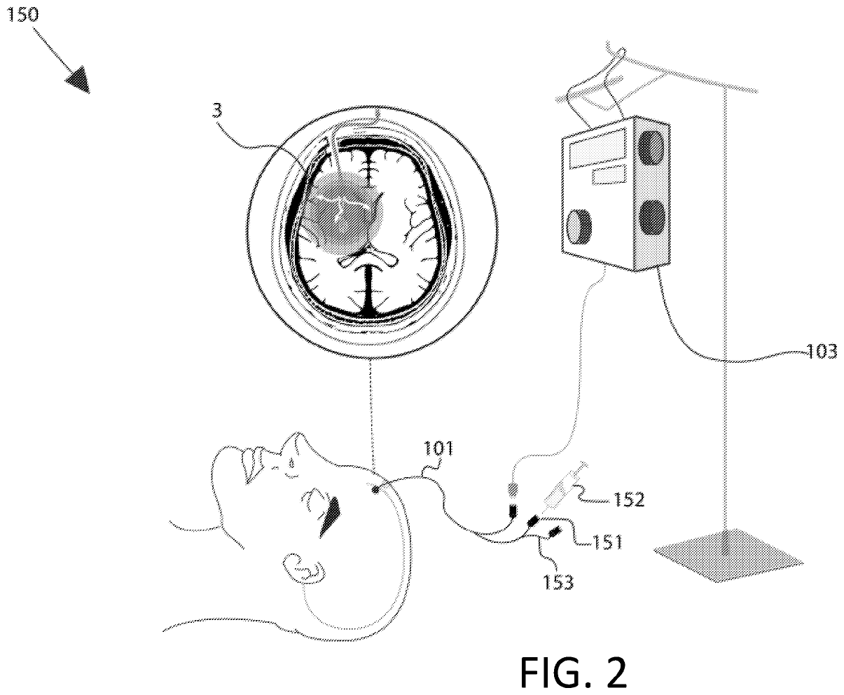

FIG. 2 illustrates a block diagram of another exemplary closed-circuit flow system with a detachable cooling system 150 that includes various additional embodiments. In this embodiment, the detachable cooling apparatus 101 includes an additional port 151 for injecting drugs or other infusion-based treatments 152, and an external ventricular drain 153 for reducing intracranial pressure. It will be understood that the port 151 can include a luer connector or the like for mating to a syringe or other delivery device for delivering an agent, such as medication. In another embodiment, the drug introduction system and the external ventricular drain 153 could be added to the thermal management and flow system 103 to allow for controlled, continuous introduction of drugs into the cavity, or automatic safety regulatory processes to account for intracranial pressure changes. The external ventricular drain 153 can include a valve or other mechanisms for control thereof. In addition, introduction of the external ventricular drain may be used to analyze many clinical factors, such as inflammatory cytokines, following surgery. It will be appreciated that the port 151 and drain 153 have dedicated lumens in the catheter structure and can be open such as at the distal end (distal tip) of the detachable cooling apparatus 101 or at another distal location along the detachable cooling apparatus 101. The system of the present disclosure thus is configured to incorporate many different instruments and functionality into a single system and therefore eliminates the need for using multiple independent instruments that must be fed through a tight opening and tunnel under the skin to burr hole in the brain.

FIGS. 3A-3G illustrate a loop embodiment of the detachable cooling apparatus 101 illustrated in FIG. 1. FIG. 3A illustrates a distal end portion 201 of a distal running catheter 202 and a proximal running catheter 204 that connect to form a loop (continuous fluid loop) that allows the cooling liquid to flow seamlessly from the distal running catheter 202 to the proximal running catheter 204. An insulated region 208 of the detachable cooling catheter is insulated through specific material selection (e.g., suitable insulation material), while the heat-exchanger region 206 is designed to promote cooling through a high-conductivity material. The heat-exchanger region 206 thus comprises a non-insulated region. Other qualities of the material are possible. In some embodiments, the loop is bent at a substantially 180-degree angle, but other angles are possible. In addition, the loop can include a soft curve to channel the liquid from one catheter to the other. The loop may be formed by fusing the distal running catheter 202 together with the proximal running catheter 204, which is illustrated in FIG. 3A as a fusion point 205. In other embodiments a single catheter is looped without kinking. The distal running catheter 202 and the proximal catheter 204 may be made from a high thermal conductivity semi-rigid material.

The distal running catheter 202 includes the cooled liquid received from the thermal management and flow system (not illustrated—See item 103, FIG. 1). The proximal running catheter 204 includes liquid that is heated when the cooled liquid is exposed to surrounding tissue in the heat-exchanger region 206. In other words, a heat exchange operation has occurred in the heat-exchanger region 206 and the cold temperatures of the cooled liquid is transferred to the tissue resulting in a cooling of the tissue and warming of the cooled liquid). In one embodiment, the heat-exchanger region (distal end portion) is cooled to a temperature of around 0° C. (by means of the cooled liquid) in order to cool the target region (target brain tissue) to a temperature of between 30° C. and 36° C. The distal running catheter 202 and the proximal running catheter 204 are covered by a portion 208 of the detachable cooling apparatus 101 that insulates the distal running catheter 202 to prevent premature temperature changes. In other words, the portion 208 comprises an insulated region defined by an outer insulation material, such as a jacket or the like, that covers at least the distal running catheter 202 to ensure the cooled liquid maintains temperature as it is delivered to the heat-exchanger region 206. Any number of different insulating materials can be used so long as they are sufficiently flexible to permit bending of this insulated region (portion 208).

The heat-exchanger region 206 may include a high conductivity material, such as a thin-walled high conductivity polyether loop that operates as a heat-exchanger region to allow the cold temperature from the cooled liquid to cool the surrounding tissue. In this example, the heat-exchanger region exposes the cooled liquid from the distal running catheter 202, which absorbs heat from the surrounding tissue. As a result, the cooled liquid becomes heated liquid and the heated liquid is transmitted outside of the patient via the proximal running catheter 204. It will be appreciated that other high conductivity materials can be used such as polyetheretherketone (PEEK), high density polyethylene (HDPE), linear low density polyethylene (LLDPE), conductive polyamide (PA), low friction polyamide-imide (PAI), polyetherketoneketone (PEKK), or conductive polyphenylene sulfide (PPS). Many of these materials can be embedded or reinforced with other conductive materials such as carbon-fiber or glass-fiber to further increase conductivity without negatively affecting imaging capabilities or device function.

FIG. 3B is a cross-sectional top view of the detachable cooling apparatus and FIG. 3C shows a second side view of the portion 201 detachable cooling apparatus 101 with additional solid lines to illustrate how the insulated region 208 covers the distal running catheter 202 and the proximal running catheter 204. FIG. 3D is a cross-sectional top view and FIG. 3E shows a third side view of the portion 201 of the detachable cooling apparatus 101 that is rotated to see only the proximal running catheter 204.

FIG. 3B illustrates possible embodiments such as a sensor 222 and an additional lumen port 224. A connector (not shown) is used to attach and detach the detachable cooling apparatus from the fluidics system and thermal management and flow system while lining up the fluidics systems distal and proximal running catheters to the detachable cooling apparatus” distal 202 and proximal 204 running catheter. In some embodiments, the connector may allow for further connections including additional lumens, guidewires, and leads. The connector may be a clip (e.g., made of metal or plastic), or a complex luer-locking mechanism. Additional lumens may be used as drug infusion ports. In some embodiments, the detachable cooling apparatus 101 may include a luminous entity for the delivery of brain photobiomodulation (PBM).

The sensor 222 may be a sensor array or a single sensor. The sensor 222 may be placed to record data anywhere along the detachable cooling apparatus 101. In some embodiments, the sensor 222 may be used to determine the temperature of the liquid as it moves from the distal running catheter 202 to the proximal running catheter 204. The sensor 222 may determine a variety of data including the temperature of the liquid, the pressure of the liquid, the intracranial pressure, the flow rate of the liquid, or other biological properties of the liquid or surrounding tissue. In some embodiments, the sensor 222 is coupled to an external controller located on the thermal management and flow system (not shown) that is operable to modify at least one of the temperature, the pressure, the flow rate, and the other biological properties. The sensor 222 and the external controller may include a mechanism for detection of a rupture within the catheter and initiating an operation to immediately inhibit further flow.

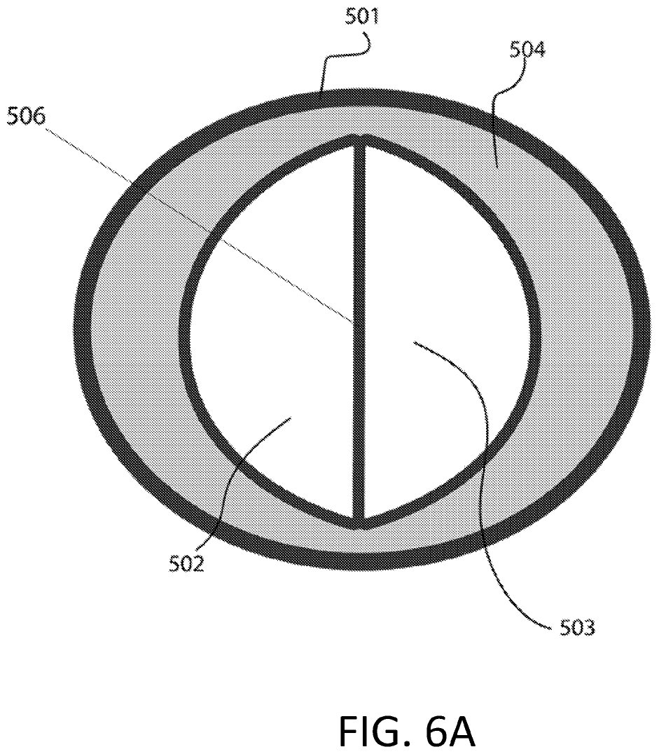

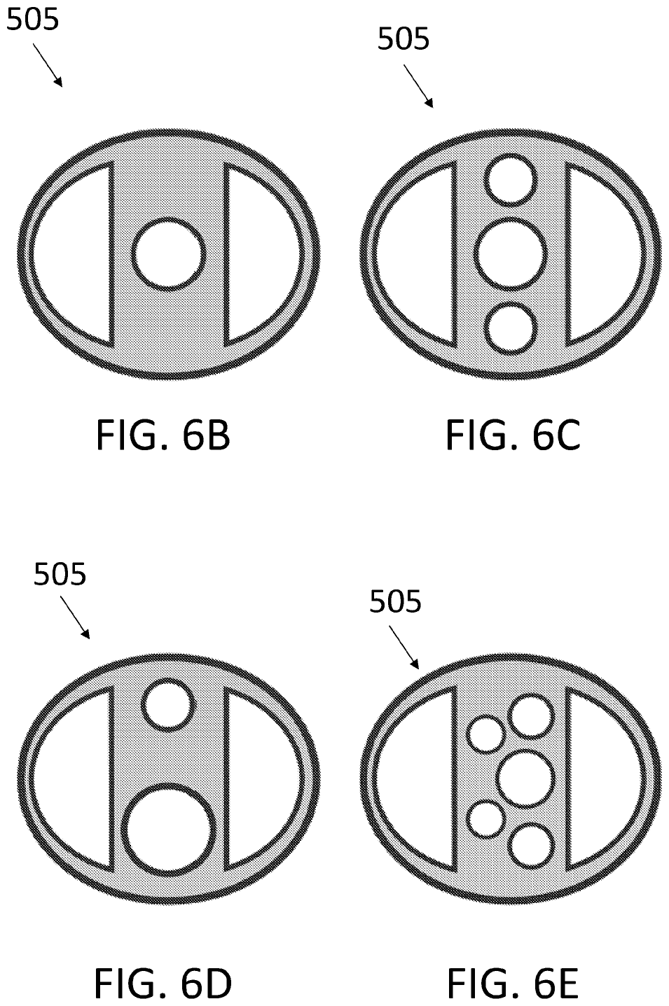

The sensor 222 can be in the form of an external intracranial pressure sensor or probe designed to monitor and detect the intracranial pressure. Intracranial pressure (ICP) monitoring uses a device (sensor or probe) placed inside the head. The monitor senses the pressure inside the skull and sends measurements to a recording device which in this case can be part of the external controller. The sensor 222 can be located at any number of different locations, such as at an interface between the insulated region 208 and heat-exchanger region 206 (FIG. 3B) or at the far distal end of the cooling apparatus or other location that is in proximity to the target site. It will be appreciated that electronics, such as wiring, etc., associated with the sensor 222 travels through one lumen that is formed as part of the multi-lumen catheter body, such as the one shown in FIGS. 6C-6E. The additional lumen port 224 may be used to administer a drug to the patient while the detachable cooling apparatus 101 is installed. Although this example illustrates the additional lumen port 224 as being between the insulated region 208 and heat-exchanger region 206, other locations are possible such as the opening of the lumen port 224 being at the far distal end of the detachable cooling apparatus 101. In one embodiment, the drug comprises an anti-inflammatory agent or a thrombolytic agent.

In one embodiment, there can be one or more internal temperature sensors for measuring the temperature of the cooling liquid in the heat-exchanger region and at least one external temperature sensor (e.g., sensor 222) for measuring the temperature of the brain tissue at a location spaced from the location of the at least one internal temperature sensor. For example, as described herein, the external temperature sensor can be located along the flexible midsection of the apparatus. The internal and external sensors are in communication with the controller and allow for temperature measurements to be recorded and displayed. In addition, the controller can be configured to take action in response to the temperature measurements. For example, the external temperature sensor is intended to measure the radial cooling effect of the apparatus and therefore, if brain tissue temperature detected by the external temperature sensor is less than desired, the temperature of the cooling liquid can be adjusted (e.g., lowered).

FIG. 3G illustrates that at least in one embodiment, the heat-exchanger region 206 is translucent and the insulated region 208 is opaque (shown with gray shading) for purposes of showing the location of the insulation, but other versions of the materials are possible.

FIGS. 4A-4G illustrate a bag embodiment of the detachable cooling apparatus 101 of FIG. 1 according to some embodiments wherein a heat-exchanger region 305 may be primarily composed of a thin, high-conductivity bag 306. FIG. 4A illustrates a distal end portion 301 of the detachable cooling apparatus 101 that illustrates a distal running catheter 304, a heat-exchanger region 305, a bag 306, and an example placement of a drug infusion port 307.

FIGS. 4B-4C are a cross-sectional top view and cross-sectional side view of the detachable cooling apparatus 101 that includes a proximal running catheter 302, the distal running catheter 304, and the bag 306. The distal running catheter 304 forms a distal opening (lumen) 311 that provides liquid as an input to the bag 306 or alternatively, the distal running catheter 304 can include one or more distal openings formed therein that are in fluid communication within an interior of the bag 306 for delivering of the cooled liquid to the interior of the bag 306. The distal running catheter 304 receives cooled liquid from the thermal management and flow system (not shown). A portion of the distal running catheter 304 is covered by an insulating region 308 that insulates the distal running catheter 304 to inhibit thermal changes in the fluid until reaching the heat-exchanger region 305. The bag 306 may be made of a material with high thermal conductivity that allows the cooled liquid to rapidly absorb heat from the surrounding tissue of the patient. The bag 306 thus constitutes a non-insulated distal region. In the illustrated embodiment, the bag 306 surrounds both the distal running catheter 304 and the proximal running catheter 302; however, it will be understood that the bag 306 can be configured to only surround the distal running catheter 304 that carries the cooled liquid to allow placement of the bag 306 against tissue that is to be cooled.

The proximal running catheter 302 forms a proximal lumen 312 that receives liquid from a proximal most point 312 of the bag 306 as an output. Alternatively, the proximal running catheter 302 can include one or more openings that provide fluid communication between the interior of the bag 306 and the inner lumen of the proximal running catheter 302. In this embodiment, a sensor 309 is integrated into the bag 306. The sensor 309 can include an array of sensors or a single sensor. The sensor 309 can exist anywhere along the detachable cooling apparatus 101 to detect a wide variety of datapoints as previously elaborated.

FIGS. 4D-4E illustrate the distal portion 301 in a different orientation showing the distal running catheter 304, the distal running catheter 304, and the bag 306. FIGS. 4D-4E show the distal portion 301 rotated 90 degrees as compared to FIGS. 4B-4C. FIG. 4G is a side perspective view of the distal portion 301.

FIG. 4G shows an embodiment in which the insulated regions are opaque (shown with gray shading)) for purposes of showing the location of the insulation and the areas of heat transfer are translucent, but other versions of the materials are possible.

FIG. 5A-5G illustrate a spiral embodiment of the detachable cooling apparatus 101 of FIG. 1 according to some embodiments. FIG. 4A illustrates a distal end portion 401 of the detachable cooling apparatus 101 that illustrates a distal running catheter 402, a proximal running catheter 404, and a heat-exchanger region 408.

FIGS. 5B-5C are top cross-sectional and side cross-sectional views of the distal portion 401 of the detachable cooling apparatus 101 that includes a distal running catheter 402, the proximal running catheter 404, a distal lumen 406, a proximal lumen 407, and the heat-exchanger region 408. The distal running catheter 402 may be made of polyurethane. The distal running catheter 402 delivers the cooled liquid to a terminal (distal) end of the detachable cooling apparatus 101 where the cooled liquid is released via a distal lumen (opening) 406 into the heat-transfer portion 408 of the proximal running catheter 404. The heat-transfer portion 408 flows into the proximal running catheter 404 via the proximal lumen (opening) 407. The spiral-shaped proximal running catheter 404 that makes up the heat-exchanger region 408 may be made of an ultra-low-durometer, high-thermal conductive material. This heat-exchanger region 408 wraps around the distal running catheter 402 and cools surrounding tissue. In other words, the cooled liquid is delivered within the linear inner lumen of the distal running catheter 402 and then as a distal end thereof, flows through the distal lumen 406 into the heat-exchanger region 408 that has a spiral construction wrapping around the distal running catheter 402. The wrapped, spiral form of the heat-exchanger region 408 provides increased surface for conducting heat transfer. Much like a radiator, once the cooled liquid flows through the spiral shaped heat-exchanger region 408, the now warmed liquid flows into the proximal running catheter 404 by flowing through the proximal lumen 407.

In some embodiments, the detachable cooling apparatus 101 includes an insulated region 416 that insulates the cooled liquid until it reaches the heat-exchanger region 408. As described above, after the cooled liquid travels through the coiled portion of the proximal running catheter 404 and absorbs heat from the surrounding tissue, the proximal running catheter 404 flows into a proximal lumen 407 to transmit liquid back to the thermal management and flow system (not shown).

FIGS. 5D-5E shows the distal portion 401 of the detachable cooling apparatus 101 rotated 90 degrees as compared to FIGS. 5B-5C. In this embodiment, the detachable cooling apparatus 101 can include a sensor 427 and an additional lumen port 429. The sensor 427 may be an array of sensors or a single sensor.

FIG. 5G illustrates one embodiment of the distal portion 401 of the detachable cooling apparatus 101 of FIG. 5A. In this example, the insulated regions are opaque (shown with gray shading) for purposes of showing the location of the insulation and the heat transfer regions are translucent, but other versions of the materials are possible.

FIGS. 6A-6E illustrates cross-sections of the fluidics midsection and alternative embodiments of the detachable cooling apparatus 101. FIG. 6A illustrates a first fluidics midsection 501 embodiment that includes a distal running lumen 502, a proximal running lumen 503, and insulation 504 for insulating the two lumens 502, 504. A space 506 indicated between the lumens 502, 503 can include additional insulation 504. The insulation 504 can thermally insulate the catheters using a high-durometer, ultra-low-thermal conductivity polyurethane to reduce line-loss. The second group of fluidics midsections shown in FIGS. 6B to 6E at 505 illustrate different lumens for alternate embodiments including drug infusion, lavage, or sensor wires. These figures show different sized lumens and different lumen locations for performing different functions. In general, the largest sized lumens relate to the distal running lumen 502 and the proximal running lumen 503. The other lumens can be associated with a drug delivery port for delivering a drug to the target site, an aspiration device, and can carry electronics, such as wires, associated with a sensor 222.

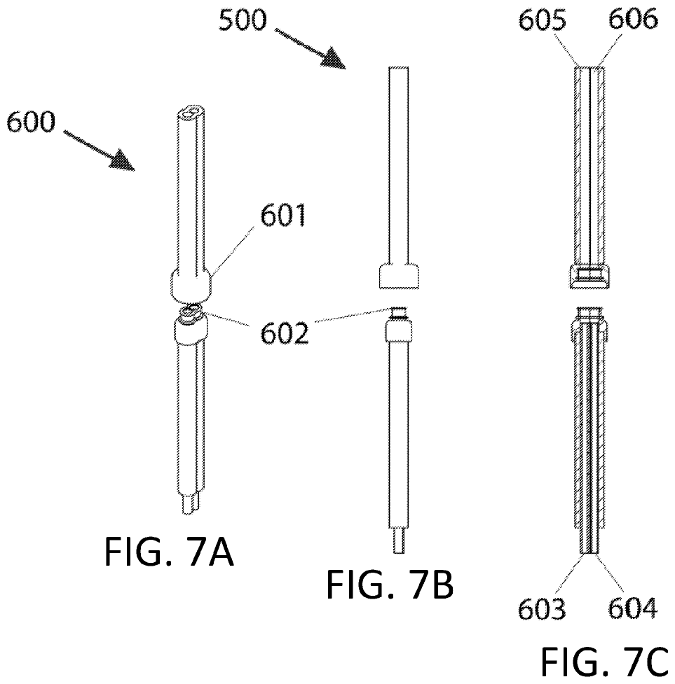

FIGS. 7A-7C illustrate one exemplary connection 600 to attach the detachable cooling apparatus 101 to the thermal management and flow system 103 according to some embodiments. In this example, a female end 601 is connected to a male end 602 wherein the distal running catheter of the detachable cooling apparatus 605 lines up with the distal running catheter of the thermal management and flow system 603, and the proximal running catheter of the detachable cooling apparatus 606 lines up with the proximal running catheter of the thermal management and flow system 604. In other words, when the connection 600 is made, fluid and sealed communication is obtained between the catheters associated with the detachable cooling apparatus 101 and those catheters associated with the thermal management and flow system 604. Other locks are possible, such as a screw lock. The connection 600 should be of a type that is easy to effectuate and easy to reverse.

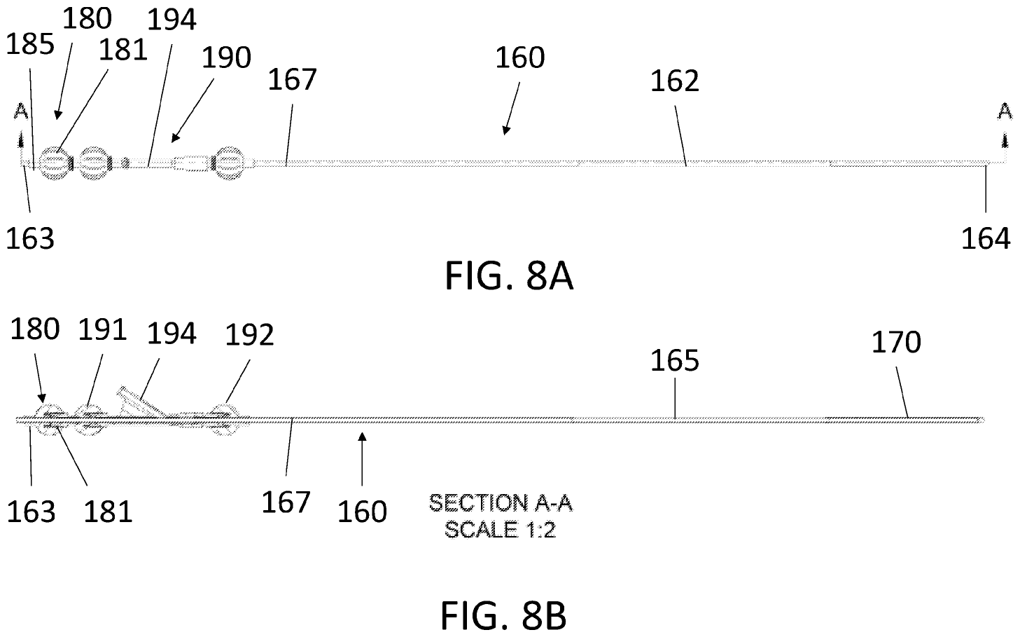

FIGS. 8A and 8B illustrate another embodiment of the detachable cooling apparatus 160 that has an elongated body 162 that terminates in a proximal end 163 and an opposing distal end 164. As shown, the distal end 164 is closed end, while the proximal end 163 can be an open end. In this embodiment, a distal end section of the detachable cooling apparatus 160 includes a heat transfer balloon 170 which acts as a heat-exchanger region in the manner described previously herein. The heat transfer balloon 170 can extend to or close to the distal end 164. As in the previous embodiments, the detachable cooling apparatus 160 includes the distal running catheter and the proximal running catheter. The distal running catheter can have a distal opening or can define a distal lumen that provides for and allows the cooled liquid to flow directly from the distal running catheter into the interior of the heat transfer balloon 170. This distal opening or distal lumen is preferably located at or near the distal end of the heat transfer balloon 170. The proximal running catheter can have a proximal opening or can define a proximal lumen that provides for and allows the liquid to flow directly out of the inside of the heat transfer balloon 170 into the proximal running catheter for return to the thermal management and flow system. The distal opening is located downstream of the proximal opening (i.e., closer to the distal end).

The distal end of the detachable cooling apparatus 160 is preferably formed of a material that is not rigid so as to minimize potential damage from the detachable cooling apparatus 160 moving while in place (at the target site).

The detachable cooling apparatus 160 includes an intermediate insulated region 165 as shown in which an insulating material covers at least the distal running catheter. This intermediate insulated region 165 is preferably flexible and as described herein is formed to allow the heat-exchanger region (e.g., balloon 170) and the intermediate insulated region 165 to undergo two separate 90° turns. At a proximal end region 167, there is no insulating material and this section serves as a rigid connection region to allow for the receipt of the cooled liquid from the thermal management and flow system and the return of the warmed liquid back to the thermal management and flow system. The proximal end region 167 can be form of a suitable rigid plastic material that is not flexible and does not collapse when a connector is securely coupled thereto. The proximal end region 167 can thus include an outflow port and an inflow port. The outflow port is in fluid communication with the inner lumen of the proximal running catheter for discharging the warmed liquid, while the inflow port is in fluid communication with the inner lumen of the distal running catheter for receiving the cooled liquid. As shown, the outflow port can be located along the side of the proximal end region 167, while the inflow port can be located at the proximal end of the proximal end region 167. The arrangement of the catheters and their respective ports and the corresponding flow path are similar to what is illustrated and described with reference to the bag embodiment of FIGS. 4A-4G. However, other locations are equally possible. The inflow and outflow ports can be in the form of holes or openings in the respective catheter.

The detachable cooling apparatus 160 can also include a first seal 180 and a second seal 190. The first seal 180 can be in the form of a first rotary seal member and the second seal 190 can be in the form of a second rotary seal member. The first rotary seal member includes a first rotatable knob (cinch element) 181 or the like that can be screwed down in order to effectively couple (attach) the first rotary seal member to the detachable cooling apparatus 160. The main body of the first rotary seal member is flexible in nature (e.g., a silicone tubular structure) and is dimensioned to be disposed about the rigid (reinforced) proximal end region of the detachable cooling apparatus 160 so as to form a seal thereto. The tightening of the first rotatable knob 181 causes a cinching against and compresses and seals the flexible main body of the first rotary seal member against the rigid proximal end region. Since the first seal 180 is attached to this rigid (reinforced) proximal end region, there internal lumens maintain their shape and do not compress. Conversely, when the first rotatable knob 18 (cinching element) is unscrewed, the first rotary seal member can be removed from the detachable cooling apparatus 160. The first rotary seal member provides an inlet port 185 at one end thereof that is placed in fluid communication with the inflow port of the detachable cooling apparatus 160 to allow cooled liquid to flow through these two ports into the inner lumen of the distal running catheter. As described herein, the ability to quickly and easily remove the first seal 180 provides a number of advantages to that allow the detachable cooling apparatus 160 to be used intracranially as discussed herein. The first seal 180 can thus be attached to a luer and both are thus located where the inflow lumen is located.

The second rotary seal member (second seal 190) is an elongated structure that has a second rotatable knob 191 or the like at one end and a third rotatable knob 192 or the like at the other end. Similar to the first rotatable knob 181, the second and third rotatable knobs 191, 192 are designed to be tightened about the body of the detachable cooling apparatus 160 to securely couple (attach) the second rotary seal member thereto. Unscrewing the second and third rotatable knobs 191, 192 allows for the easily uncoupling (removal) of the second rotary seal member. The second rotary seal member also has a side outflow port 194 that is located along the side of the second rotary seal member. The side outflow port 194 can be in the form of a leg that extends radially outward from the side of the second rotary seal member. The second rotary seal member can be disposed about and is sealingly coupled to the proximal end region 167 and/or the intermediate insulated region 165. The second rotary seal member also covers the outflow port associated with the proximal running catheter and is designed so that warm fluid flowing through the outflow port flows into the interior of the secondary rotary seal member and exits through the side outflow port 194. The rotatable knobs 191, 192 thus isolate the side outflow port 194. As shown, the second rotary seal member is thus disposed between the first rotary seal member and the distal end 164. It will be understood that in the construction, an outflow luer can be connected to the outflow port and connected to the proximal seal (180).

The second seal 190 can thus be attached on the midpoint of the detachable cooling apparatus distal to the outflow port. The two seals 180, 190 thus isolate a midpoint outflow port and a proximal inflow port by using 3 seals (2 to isolate the outflow port and one to isolate the inflow port).

It will also be appreciated that the heat transfer balloon can be of a type that can be inflated/deflated using traditional techniques such as delivery of a fluid. In the disclosed embodiment, the inflation media of the heat transfer balloon is the cooled liquid that flows within the distal running catheter to the heat transfer balloon.

The first seal 180 and the second seal 190 provide a rotary action to the device and this allows different movements of the device, thereby allowing maneuvering of the device to properly position the device at the target location.

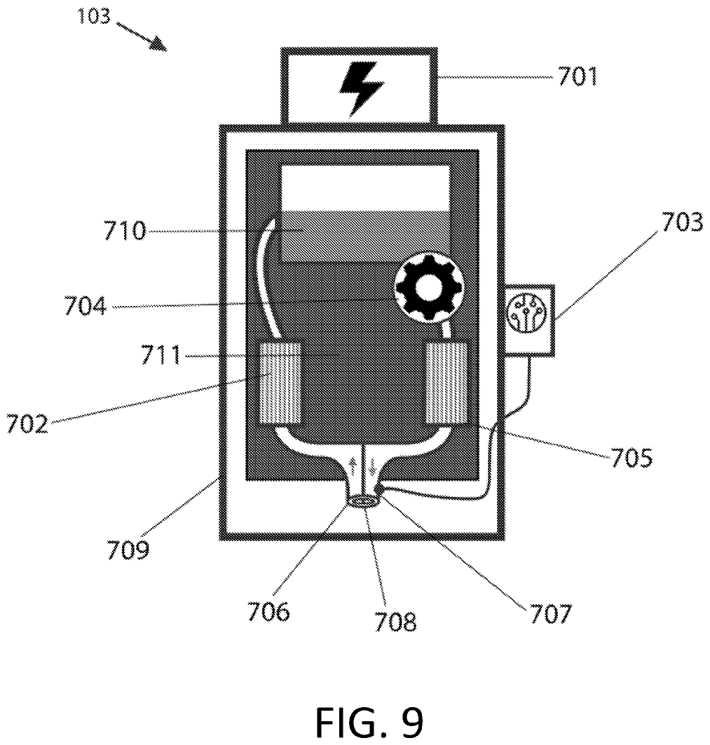

FIG. 9 illustrates a block diagram of a thermal management and flow system 103 according to some embodiments. The thermal management and flow system 103 induces continuous flow of liquid coolant through pressure induced by a pump 704. Liquid enters the thermal management and flow system 103 through an inflow port 706 and is pumped through a cooled flow-through system 702, which may include high thermal-conductivity capillary tubes, and into a thermally controlled reservoir 710. Liquid is then moved through a pump 704 (e.g., one or more pumps), and exits through another cooling flow-through system 705 which may contain a series of cooled capillary tubes 705 into the insulated midsection via an outflow port 708. A Peltier-type thermoelectric stainless-steel liquid cooler or cold plate cooler with an attached stainless steel liquid block may be used to induce cooling 711. Sensors 707 attached from the inflow port 706 or outflow port 708 may report to a controller 703 to alter temperature, flow, drug delivery and other rates (e.g., flow rate) accordingly. The controller 703 may also utilize data collected from other sensors on the detachable cooling apparatus 101 or other areas of the system. A power supply 701 is integrated into the thermal management and flow system to allow for the thermal management and flow system 103 to be completely mobile. The thermal management and flow system 103 can be enclosed in a housing 709 integrating each component. The thermal management and flow system 103 thus incorporates a control feedback loop in which the user can input a desired cooling temperature for the cooled liquid and parameters, such as flow rate of the cooled liquid, are monitored and controlled in order to achieve the desired level of cooling at the target site.

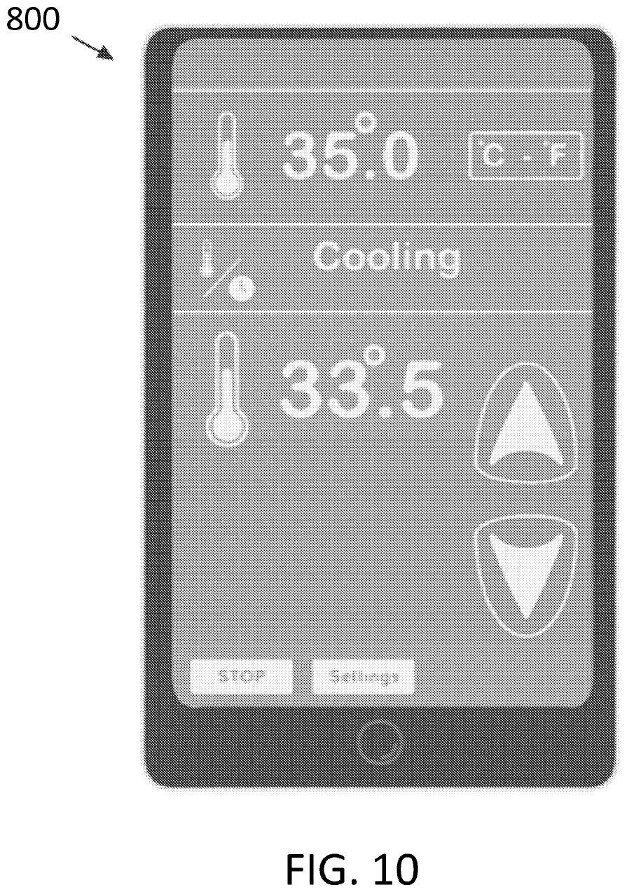

FIG. 10 illustrates one exemplary user interface 800 for managing the detachable cooling apparatus according to some embodiments. In this example, the user interface includes a current temperature of the liquid in the detachable cooling apparatus 101 as 35.0 degrees F. and a desired temperature of 33.5 degrees F. As mentioned previously, there are two temperatures of significance with one being the actual temperature of the cooled liquid and the other being the temperature of the target site (i.e., the surrounding tissue). The user interface 800 can display the current measured temperature of the cooled liquid and the inputted temperature of the cooled liquid. The user interface 800 also includes buttons (inputs) for changing the desired temperature of the liquid, a button for stopping the thermal management and flow system 103 from cooling the liquid, and a button for modifying the settings, such as monitoring the operation of the detachable cooling apparatus 101, activate manual or automatic rewarming, and power off the thermal management and flow system 103. It will be appreciated that the user interface 800 can be part of a computing device, such as a laptop, desktop, etc., or can be part of a mobile device, such as an app on a cellular phone, tablet, etc. Alerts, both visual and auditory, can be incorporated into the software that is executed to alert the user as to an event that needs attention (e.g., the measured temperature is outside of an acceptable range).

As shown and described herein, the detachable cooling apparatuses disclosed herein include three main regions, namely, a distal end region which serves as the heat-exchanger region, an intermediate flexible region that is an insulated region, and a rigid proximal end region which is a stiffer, reinforced region that defines the section to which one or more connectors are attached. The different regions that can be formed of different materials can be coupled to one another using conventional techniques, such as use of bonding agents, welds, etc.

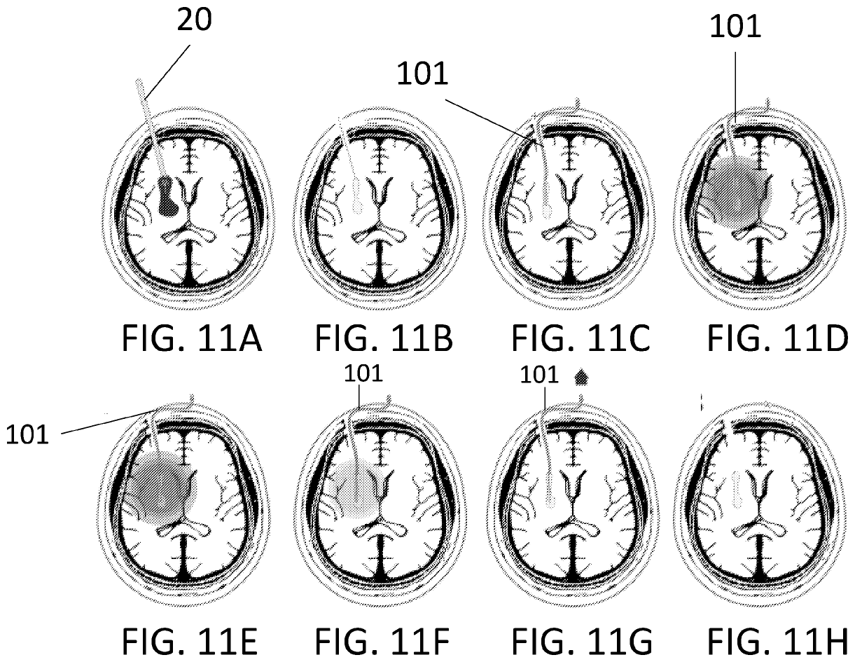

FIGS. 11A-11H show the brain (i.e., the target area) during deployment, operation, and removal of the detachable cooling apparatus 101 according to some embodiments. FIGS. 11A-11H thus illustrate successive steps that are performed. It is well understood that during neurosurgery, the required instruments and tools are delivered to the target site using a delivery device, such as a SurgiScope. A SurgiScope is a microscope and robot designed to hold tools and assist in positioning those tools during neurosurgery. The unit can be mounted on the ceiling and can hold instruments such as endoscopy tools, biopsy needles, and electrodes. The associated software allows for target and trajectory determination. In a first step, illustrated in FIG. 11A, a hemorrhage evacuation is performed and the hemorrhage is removed from the brain 10 using a traditional tool, such as an aspiration device 20. In a second step, illustrated in FIG. 11B, the cavity is seen, with the aspiration device 20 removed but the approach (i.e., an opening passing through the skull) still accessible by future devices. In a third step, illustrated in FIG. 11C, the detachable cooling apparatus 101 is deployed within the cavity via the same burr hole utilized for evacuation (step 1). The detachable cooling apparatus 101 is tunneled away from the burr hole, allowing for the primary entry point in the brain 10 of the patient to be closed. It will be understood that the detachable cooling apparatus 101 is fed through a device, such as the SurgiScope, in order to reach the target location in the brain. In a fourth step, illustrated in FIG. 11D, cooling is initiated using the detachable cooling apparatus 101 while the patient is still in the operating room under the guidance of a surgeon. In a fifth step, illustrated in FIG. 11E, cooling is maintained between several hours to the entire length of the hospital stay. It will be appreciated that during this time, the cooling liquid continuously flows through the system and is recooled, etc., to continuously cool the target tissue. In a sixth step, illustrated in FIG. 11F, controlled rewarming is initiated prior to removal of the detachable cooling apparatus 101. In a seventh step, illustrated in FIG. 11G, upon completion of rewarming, flow is stopped, and the detachable cooling apparatus 101 is removed at the bedside using a mild pulling force (without requiring additional equipment). As described herein, in some embodiments, the proximal end connector is removed prior to removal. The controlled rewarming can occur by heating the liquid flowing through the detachable cooling apparatus 101. In an eighth step, illustrated in FIG. 11H, the tunneled exit point is closed.

The detachability of the cooling apparatuses described herein is important since the connector equipment associated with the detachable cooling apparatus is too large to pass through the lumen of the SurgiScope and similarly, during the process described above, when it is desired to remove the SurgiScope from the detachable cooling apparatus, the SurgiScope cannot pass over the connector equipment. Thus, in the case of using the seals 180, 190 (FIGS. 8A and 8B), these seals 180, 190 can be removed from the detachable cooling apparatus to allow for removal of the SurgiScope over the detachable cooling apparatus. Thus, the modularity and detachability (quick connect/quick disconnect) of the present system permits its use in an intracranial setting (neurosurgery). It will be appreciated that the other types of connectors, such as luer connectors, described herein also permit the quick attachment/detachment.

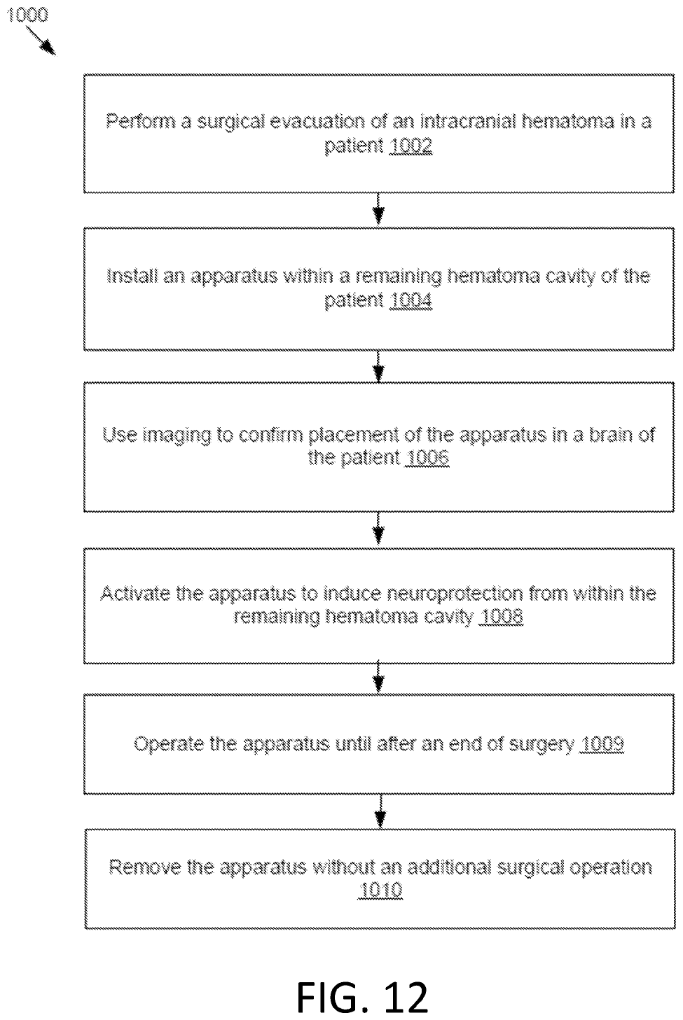

FIG. 12 illustrates a flow diagram 1000 of a method for deploying an apparatus within an ICH evacuation cavity. In some embodiments, the detachable cooling apparatus 101 of FIG. 1 is used, but other devices may be used that induce neuroprotection from within the cavity. At block 1002 (first step), a surgical evacuation of an intracranial hematoma in a patient is performed. For example, the surgical evacuation may be a minimally-invasive process of removing the IHC from the patient via a burr hole in the skull. At block 1004 (second step), an apparatus is installed within a remaining hematoma cavity of the patient. The apparatus may be the detachable cooling apparatus 101 of FIG. 1 or another device. At block 1006 (third step), imaging is used to confirm placement of the apparatus in a brain of the patient. At block 1008 (fourth step), the apparatus is activated to induce neuroprotection from within the remaining hematoma cavity. For example, the apparatus may be attached to a thermal management and flow system 103 as illustrated in FIG. 1 that includes a pump for providing cooled liquid through the apparatus that cools the tissue inside the brain. As mentioned previously, the pump is part of a flow management control system that is configured to maintain the temperature of the cooled liquid within a target range. As a result, flow rate of the cooled liquid is controlled so that a residence time of the cooled liquid in the heat-exchanger region results in the temperature of the cooled liquid within the heat-exchanger region being maintained in this desired temperature range (e.g., around freezing (32° F.)). At block 1009 (fifth step), the apparatus is operated until after an end of surgery. At block 1010 (sixth step), the apparatus is removed without an additional surgical operation. The apparatus may be removed following neuroprotective treatment. For example, the apparatus may be removed after the temperature in the remaining hematoma cavity is substantially similar to the temperature of the other tissue in the brain of the patient.

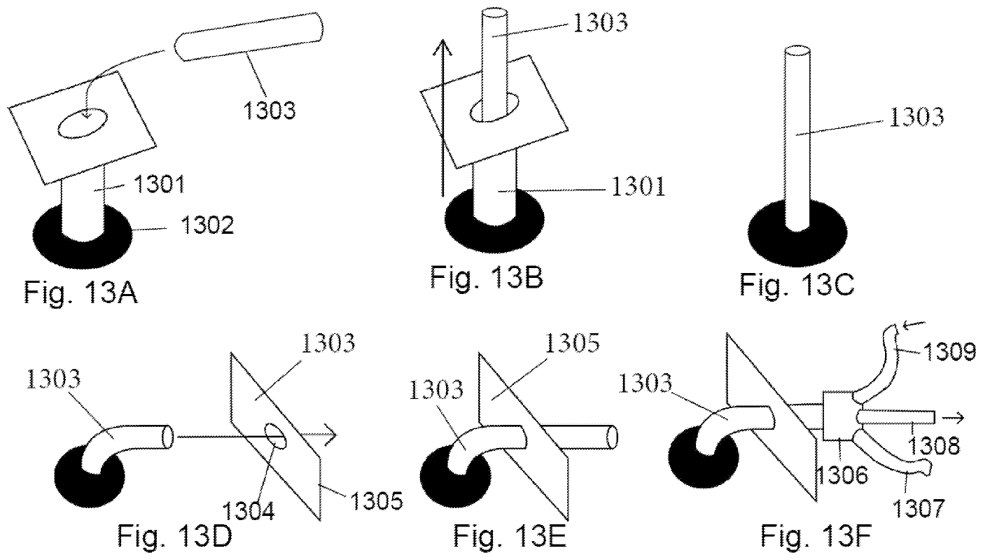

FIGS. 13A-13F illustrates the system of catheter tunneling in ICH procedures. The detachable cooling device 1303 is introduced through a SurgiScope or similar device 1301 into the brain via a burr hole 1302 (a small hole made in the skull). The SurgiScope 1301 is then removed around the catheter (device 1303) (FIG. 13B, FIG. 13C). The catheter (device 1303) is then tunneled through the skin 1305 through a hole 1304 cut with a metal stylet (FIG. 13D, FIG. 13E). After tunneling, the connectors 1306-1309 are attached to the catheter to allow for multiple luer connections (FIG. 13F). As described herein, the connectors permit attachment to various external equipment, such as system 103, sources of medication, aspiration equipment, etc.

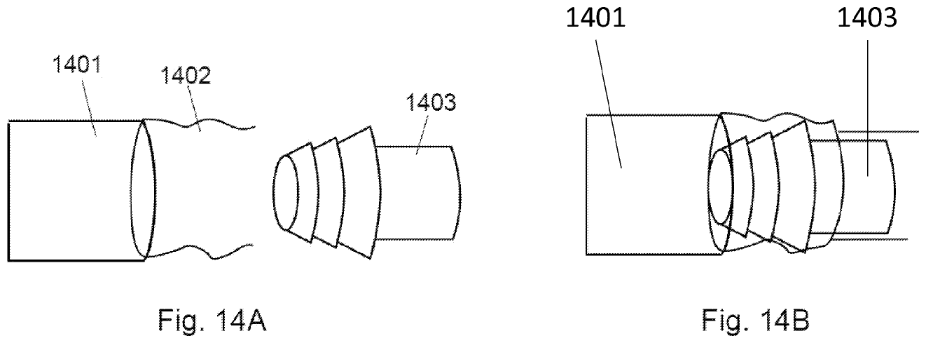

FIG. 14A illustrates a side view illustrating the proximal-most portion of the detachable cooling apparatus 1401, with an additional embodiment to allow for easy attachment to a standard metal stylet 1403 used for tunneling in neurosurgical procedures. A stylet connector 1402 could be a permanent or removable catheter section and is comprised of a stretchable material, such as silicone. FIG. 14B demonstrates the slide-over mechanism of attachment that connects these two parts together.

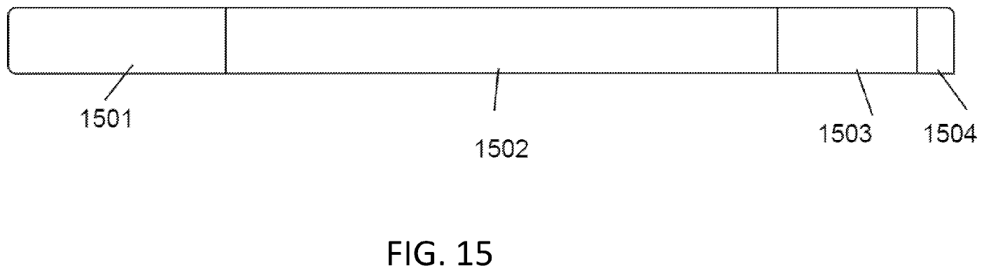

FIG. 15 depicts the various sections of one exemplary detachable cooling apparatus. The distal end comprises a heat exchanger region 1501 built with highly-conductive plastics. The midsection 1502 is a flexible, insulating region that traverses the brain, skull and skin. The proximal portion of the catheter is reinforced to allow for the use of rotator seals or other mechanisms to attach the luer connector(s) 1503. An additional embodiment designed to attach the detachable cooling apparatus to a standard metal stylet (See, FIG. 14A), generally shown at 1504, for tunneling is also depicted.

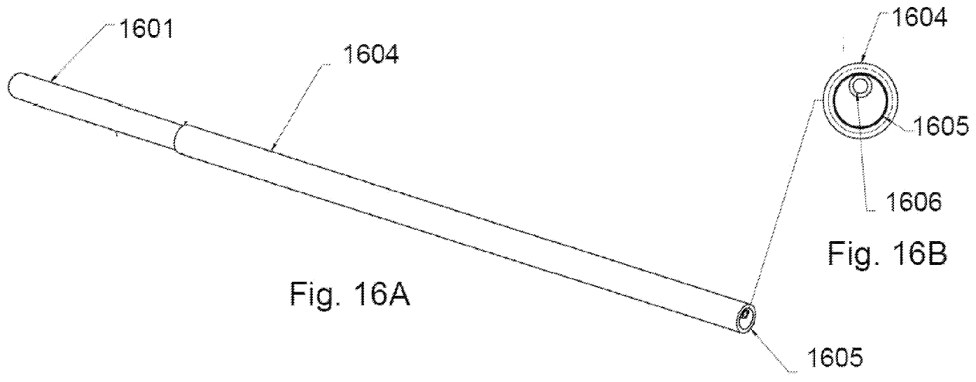

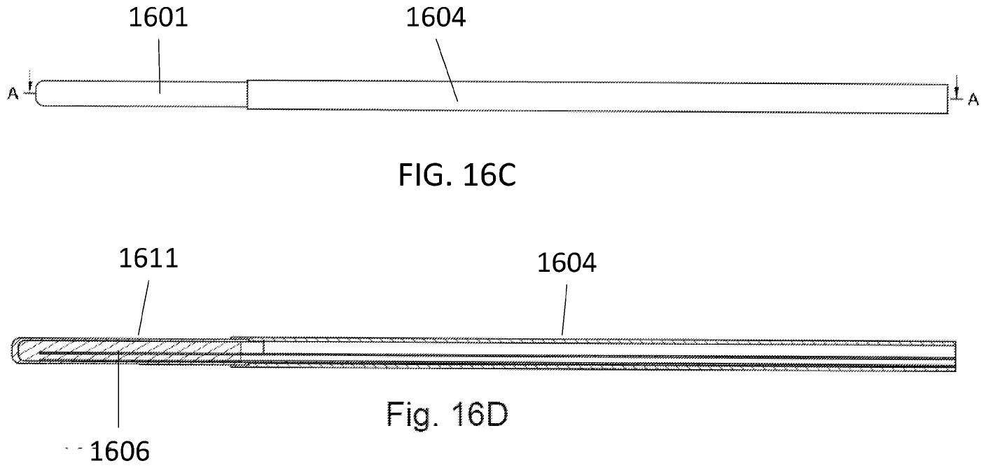

FIGS. 16A-16D illustrate possible catheter embodiments. FIG. 16B shows a cross-section of the catheter, with the inflow lumen 1606 running internal to the outflow lumen 1605 that is encased in the thermally insulating midsection 1604. The structure defining the inflow lumen 1606 can be considered to be a distally extending catheter, while the structure defining the outflow lumen 1606 can be considered to be a proximally extending catheter. The inflow lumen extends through the heat-exchanger region 1601 but ends proximal to the end of the heat exchanger region 1601. The cooled liquid thus exits the open distal end of the outflow lumen 1605 and flows into the heat-exchanger region 1601 before flowing in an opposite direction in the inflow lumen 1606. As mentioned herein, the heat-exchanger region 1601 is formed of a different material than the flexible midsection (intermediate section) 1604. In addition, the relative sizes of the inlet and outflow lumens can be different; however, the inflow lumen typically has a smaller diameter than the outflow lumen.

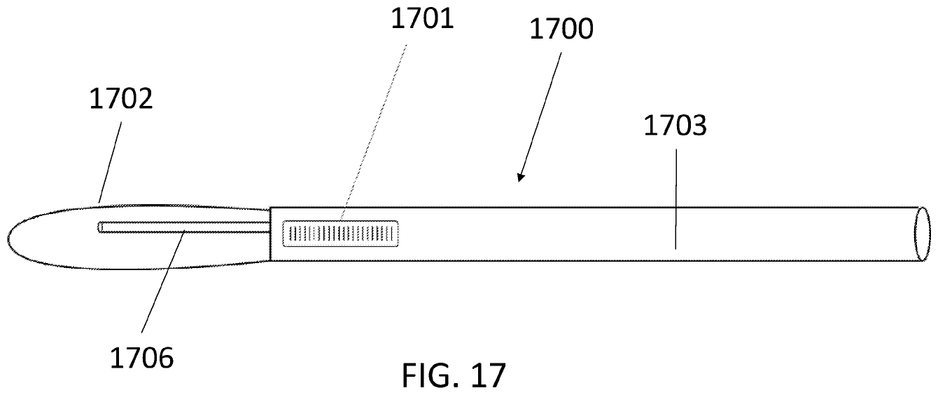

FIG. 17 illustrates an embodiment of a detachable cooling apparatus 1700 with the addition of a pressure (e.g., intracranial pressure sensor) or temperature sensor 1701 located just proximal to the heat-exchanger region 1702 which is shown as being an inflatable member, such as a bag or balloon. These sensor(s) 1701 can be placed anywhere along the flexible midsection 1703 (the flexible intermediate region). As in the embodiment shown in FIG. 16D, the apparatus 1700 includes an inflow lumen 1706 (distally running catheter) for delivering the cooled liquid to the heat-exchanger region 1702.

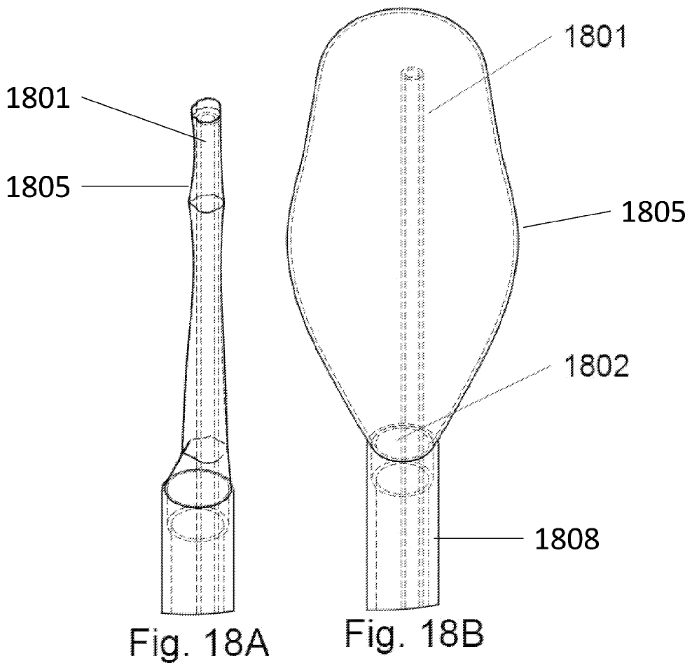

FIGS. 18A and 18B show the same/similar catheter design as in FIG. 17 with a possible embodiment of a distal heat-exchanger inflatable member 1805 (e.g., a bag or balloon). The deflated bag (FIG. 18A) is flush against the inflow port 1801. As flow is initiated, the bag 1805 inflates (FIG. 18B) to fill the cavity space within the brain. The inflow port 1801 does not extend the entire length of the bag 1805, ensuring that, when inflated, the semi-rigid inflow port is not in direct contact with the brain. The distal end of the bag is thus unsupported since the distal end of the inflow port 1801 is spaced from the distal end of the bag 1805. This allows the distal end to have some compression due to any brain tissue movement. The inflow port 1801 and outflow 1802 are sized to allow for a specific internal pressure within the bag. The flexible midsection is shown at 1808.

The system described herein is thus configured to provide localized cooling of a target area and unlike conventional intravascular cooling systems, the present system is of an extravascular nature. As mentioned herein, the cooling apparatuses described herein are designed to fit through current instrument delivery systems, such as a SurgiScope device and therefore, at least according to one embodiment, the diameter of the detachable cooling apparatus (catheter structure) is less than 4.7 mm and can be less than 4.0 mm, and can in one exemplary embodiment be between 2.0 mm and 4.0 mm to allow for tunneling under the skin. In one embodiment, the axial length of the heat-exchanger region can be between 1 cm and 3 cm. In the event of using an external temperature sensor and an internal temperature sensor, as described herein, the distance between the internal and external temperature sensors can be up to 3 cm (e.g., between 1-3 cm). In addition, in at least one embodiment, the outflow (proximally running) lumen can be slightly smaller than the inflow (distally running) lumen to ensure inflation of the inflatable member. This results in the outflow lumen being the rate-limiting step for flow rate. In one embodiment, the outer diameter (OD) is 4 mm, meaning the inner diameter (ID) of the outflow lumen is substantially smaller (<1 mm). In one embodiment, it is desirable to achieve a ˜1 mm (5F) outflow ID. In one embodiment, the catheter length (i.e., length of the detachable cooling apparatus) can be between 5 to 20 cm, such as ˜15 cm. As also discussed herein, the detachable cooling apparatus (catheter) is configured to be flexible enough to undergo two separate 90° turns due to the construction of the flexible midsection. This is similar to common EVD catheters, which are commonly made with silicone tubing (e.g., the thermally insulating outer catheter of the detachable cooling apparatus can thus be made with a flexible material such as silicone).

In the above description, for purposes of explanation, numerous specific details are set forth in order to provide a thorough understanding of the specification. It will be apparent, however, to one skilled in the art that the disclosure can be practiced without these specific details. In some instances, structures and devices are shown in block diagram form in order to avoid obscuring the description.

Reference in the specification to “some embodiments” or “some instances” means that a particular feature, structure, or characteristic described in connection with the embodiments or instances can be included in at least one implementation of the description. The appearances of the phrase “in some embodiments” in various places in the specification are not necessarily all referring to the same embodiments.

What is claimed is:

1. A detachable cooling apparatus for use in a focal hypothermia-inducing fluidics system for use in a brain of a patient comprising:
a distal running catheter forming a distal lumen that provides liquid as an input; and
a proximal running catheter forming a proximal lumen that receives the liquid as an output, wherein the proximal running catheter is fluidly coupled to the distal running catheter along a closed-circuit flow path;
wherein the distal running catheter and the proximal running catheter define a catheter body having a proximal end region, a distal end region and a flexible intermediate region between the proximal end region and the distal end region, wherein the flexible intermediate region is configured to navigate through two 90 degree turns without kinking.

2. The detachable cooling apparatus of claim 1, further comprising:
a heat-exchanger region at the distal end region, the heat-exchanger region comprising a high conductivity material for cooling surrounding tissue with the liquid that is received from the distal running catheter.

3. The detachable cooling apparatus of claim 1, further comprising:
a connector at the proximal end region, the connector being configured for attachment, detachment, and alignment of the distal running catheter and the proximal running catheter to line up to one or more catheters associated with a fluidics pump and thermal-regulating mechanism that is configured to deliver the liquid to the detachable cooling apparatus, the proximal end region being formed of a stiffer material compared to both the flexible intermediate region and the distal end region.

4. The detachable cooling apparatus of claim 1, further comprising:
a sensor array that is operable to determine at least one of a temperature of the liquid or surrounding tissue, a pressure of the liquid or surrounding tissue, a flow rate of the liquid or surrounding tissue, and biological properties of the liquid or surrounding tissue; and
an external controller operable to modify the at least one of the temperature, the pressure, and the flow rate.

5. The detachable cooling apparatus of claim 4, wherein the sensor array is located at a distal end section of the distal running catheter.

6. The detachable cooling apparatus of claim 1, further comprising:
a heat-exchanger region located in the distal end region and comprising a high conductivity loop that cools surrounding tissue with the liquid, the loop directly flow from the distal running catheter to the proximal running catheter.

7. The detachable cooling apparatus of claim 6, wherein the distal running catheter and the proximal running catheter are parallel to one another and the high conductivity loop is configured to change a flow direction of the liquid by a 180-degree angle.

8. The detachable cooling apparatus of claim 1, further comprising:
a heat-exchanger region located in the distal end region and defined by the proximal running catheter which has a high conductivity coiled form that wraps around the distal running catheter and cools surrounding tissue with the liquid.

9. The detachable cooling apparatus of claim 1, further comprising:
a heat-exchanger region located in the distal end region comprising a high conductivity bag that receives the liquid from the distal running catheter as the input to a distal region of a bag; and
wherein the proximal lumen receives the liquid from the bag as the output at a proximal region of the bag that connects the distal running catheter to the proximal running catheter and cools surrounding tissue with the liquid.

10. The detachable cooling apparatus of claim 9, wherein the flexible intermediate region comprises a fluidics midsection that thermally insulates the liquid that is input into the distal running catheter until it reaches the heat-exchanger region.

11. The detachable cooling apparatus of claim 10, wherein the fluidics midsection includes a polyurethane section that thermally insulates the liquid.

12. The detachable cooling apparatus of claim 1, further comprising an additional set of lumens and ports for receiving at least one of a drug, a lavage, ventricular drain, or sensor wires.

13. The detachable cooling apparatus of claim 12, further comprising:
a connection that attaches the proximal end region of the detachable cooling apparatus to a fluidics cooling and pump system; and
wherein the additional set of lumens and portions includes at least one lumen that introduces the drug, lavage, drain or sensor through a distal port located anywhere between the connection and a distal-most end of the detachable cooling apparatus.

14. The detachable cooling apparatus of claim 13, wherein the connection is one of a screw lock and a luer lock.

15. The detachable cooling apparatus of claim 1, wherein a diameter of the detachable cooling apparatus is less than 4.7 mm.

16. The detachable cooling apparatus of claim 2, further including an internal temperature sensor within the heat-exchanger region for measuring a temperature of the liquid and an external temperature sensor for measuring a temperature of the surrounding tissue.

17. The detachable cooling apparatus of claim 16, wherein the external temperature sensor is located along the flexible intermediate region and is located up to 3 cm from the internal temperature sensor.

18. A focal hypothermia-inducing fluidics system for cooling a target location in a brain of a patient comprising:
a thermal management and flow system (TMFS) that is operable to alter a liquid to a specific temperature and to regulate a flow rate of the liquid, the TMFS including a pump for moving the liquid through the TMFS, an inflow port that receives the liquid, a cooling unit to cool the liquid, and an outflow port for discharging the liquid;
a closed-circuit flow system with a detachable cooling apparatus that is detachably coupled to the inflow port and the outflow port of the TMFS and includes a distal running catheter that receives cooled liquid from the TMFS and a proximal running catheter that returns the liquid to the TMFS; and
at least one distal sensor that is operable to determine at least one of a temperature, a pressure, the flow rate, and a biological property of at least one of the liquid and a surrounding region at the target location within the brain.

19. The focal hypothermia-inducing fluidics system of claim 18, wherein the TMFS further comprises:
a cooling unit that physically contacts a plurality of capillary tubes to increase or decrease the temperature of the liquid, wherein the cooling unit operates using at least one of peltier cooling, liquid cooling, evaporative cooling, and passive cooling.

20. The focal hypothermia-inducing fluidics system of claim 18, wherein the pump of the TMFS is at least one of a positive displacement pump, a rotary-type pump, a gear pump, a screw pump, a peristaltic pump, a rotary vane pump, a centrifugal pump, an impulse pump, a hydraulic ram pump, a pulser, an airlift pump, a velocity pump, a radial-flow pump, and a centrifugal and axial-flow pump.

21. The focal hypothermia-inducing fluidics system of claim 18, further comprising:
a controller that is operable to communicate with a sensor array to regulate temperature and flow rate of the liquid.

22. The focal hypothermia-inducing fluidics system of claim 18, wherein the controller performs at least one of automatic detection of a rupture within a line and includes an automatic kill switch that stops power to the thermal management and flow system in response to a rupture detection the line.

23. The focal hypothermia-inducing fluidics system of claim 18, wherein the detachable cooling apparatus includes a heat-exchanger region, and further comprising:
a fluidics midsection that thermally insulates the liquid that is input into the distal running catheter from the TMFS until it reaches the heat-exchanger region.

24. The focal hypothermia-inducing fluidics system of claim 23, wherein the fluidics midsection includes a polyurethane section that thermally insulates the liquid.

25. The focal hypothermia-inducing fluidics system of claim 18, wherein the detachable cooling apparatus comprises an elongated body with an exposed distal tip region that acts as a heat-exchanger region.

26. The focal hypothermia-inducing fluidics system of claim 25, wherein the distal running catheter and the proximal running catheter are parallel to one another and are fluidly connected at distal ends thereof for transferring the liquid flowing within the distal running catheter to the proximal running catheter for return to the TMFS.

27. The focal hypothermia-inducing fluidics system of claim 25, wherein the proximal running catheter has a coiled section that is coiled about the distal running catheter, the proximal running catheter defining the heat-exchanger region.

28. The focal hypothermia-inducing fluidics system of claim 27, wherein a distal end of the distal running catheter includes a distal opening that opens into the proximal running catheter for delivering the liquid to the coiled section and the proximal running catheter includes a proximal opening for receiving the liquid from the coiled section and for delivering the liquid back to the TMFS.

29. The focal hypothermia-inducing fluidics system of claim 18, further including a a heat-exchanger region comprising a high conductivity bag that receives the liquid from the distal running catheter as an input to a distal region of the bag; and
wherein the proximal running catheter receives the liquid from the bag as an output at a proximal region of the bag, the bag defining a flow path that connects the distal running catheter to the proximal running catheter and cools the surrounding tissue with the liquid.

30. The focal hypothermia-inducing fluidics system of claim 18, further comprising a set of lumens and ports that provide at least one of a drug, a lavage, ventricular drain, or sensor wires.

31. The focal hypothermia-inducing fluidics system of claim 18, further comprising a drug infusion port and drug infusion lumen that is open along the detachable cooling apparatus for cooling the target location of the brain.

32. The focal hypothermia-inducing fluidics system of claim 18, further comprising a user interface that is operable to provide a user with an option for changing a temperature or flow rate of the liquid in the system.

33. The focal hypothermia-inducing fluidics system of claim 18, wherein the distal sensor array comprises an intracranial pressure sensor that is operable to determine an intracranial pressure within the brain.

34. The focal hypothermia-inducing fluidics system of claim 33, wherein the intracranial pressure sensor is located in or adjacent a heat-exchanger region located at a distal end of the detachable cooling apparatus.

35. The focal hypothermia-inducing fluidics system of claim 18, wherein the detachable cooling apparatus includes a rigid proximal end portion that includes an inflow port and an outflow port, a first seal being sealingly coupled to the rigid proximal end portion at a proximal end thereof and a second seal being sealingly coupled to the rigid proximal end portion at a location between the first seal and a flexible midsection of the detachable cooling apparatus, the second seal including a side port that is in fluid communication with the outflow port that is part of the detachable cooling apparatus.

36. The focal hypothermia-inducing fluidics system of claim 35, wherein the first seal comprises a first rotary seal and the second seal comprises second and third rotary seals with the outflow port and side port being located between the second and third rotary seals.

37. A method for deployment of a detachable cooling apparatus intracranially using the focal hypothermia inducing fluidics system of claim 18 following intracranial hemorrhage evacuation.

38. A method for deployment of a detachable cooling apparatus intracranially using the focal hypothermia inducing fluidics system of claim 18 following at least one of intracerebral hemorrhage (ICH) evacuation, craniectomy, and intraparenchymal operations.

39. The method of claim 38, wherein a proximal section of the detachable cooling apparatus remains external to a cranium of a patient and is tunneled beneath skin of the patient.

40. A method for treating symptoms associated with intracerebral hemorrhage (ICH), craniectomy-requiring surgeries, or intraparenchymal operation comprising the steps of:
performing a surgical evacuation of an intracranial hematoma in a brain of a patient;
installing an apparatus within a remaining hematoma cavity of the patient;
confirming placement of the apparatus in the remaining hematoma cavity of the brain of the patient;
activating the apparatus to induce neuroprotection from within the remaining hematoma cavity;
operating the apparatus until after an end of surgery; and
removing the apparatus without an additional surgical operation.

41. The method of claim 40, wherein the step of installing the apparatus comprises the step of tunneling the apparatus under skin and navigating the apparatus through two 90 degree turns without kinking.

42. The method of claim 40, wherein the step of confirming comprises the step of using imaging or direct visualization of the apparatus using an endoscope.

43. The method of claim 40, wherein the step of inducing neuroprotection comprises inducing focal hypothermia.

44. The method of claim 40, further comprises the step of coupling a connector to a proximal end of the apparatus prior to the step of activating the apparatus.

45. The method of claim 40, further including the step of using the apparatus to deliver a neuroprotector agent to the remaining hematoma cavity.

46. The method of claim 43, further including the steps of:
measuring a temperature of cooled liquid that circulates in a heat-exchanger region of the apparatus using an internal temperature sensor; and
measuring a temperature of tissue of the brain using an external temperature sensor.

47. The method of claim 40, wherein the apparatus includes a closed-circuit flow system with a detachable cooling apparatus that includes a distal running catheter that receives cooled liquid; a proximal running catheter and a heat-exchanger region at distal end section of the detachable cooling apparatus, the heat-exchanger region comprising a high conductivity material for cooling surrounding tissue with the liquid that is received from the distal running catheter.

48. The method of claim 47, further comprising a thermal management and flow system (TMFS) that is operable to alter the liquid to a specific temperature and to regulate a flow rate of the liquid, the TMFS including a pump for moving the liquid through the TMFS, an inflow port that receives the liquid, a cooling unit to cool the liquid, and an outflow port for discharging the liquid, the outflow being fluidly coupled to the distal running catheter, the inflow port being fluidly coupled to the proximal running catheter.

49. The method of claim 48, further comprising a distal sensor array that is operable to determine at least one of a temperature, a pressure, the flow rate, and a biological property of the liquid and a surrounding region at the target location.

50. The method of claim 47, wherein the step of removing the apparatus comprises the step of removing the detachable cooling apparatus by pulling a proximal end of the detachable cooling apparatus outward from a skull of the patient.

51. The method of claim 48, wherein the distal running catheter and the proximal running catheter are parallel to one another and are fluidly connected at distal ends thereof for transferring the liquid flowing within the distal running catheter to the proximal running catheter for return to the TMFS.

52. The method of claim 47, wherein the proximal running catheter has a coiled section that is coiled about the distal running catheter, the proximal running catheter defining the heat-exchanger region.

53. The method of claim 48, wherein a distal end of the distal running catheter includes a distal opening that opens into the proximal running catheter for delivering the liquid to the coiled section and the proximal running catheter includes a proximal opening for receiving the liquid from the coiled section and for delivering the liquid back to the TMFS.

54. The method of claim 48, wherein the heat-exchanger region comprises a high conductivity bag that receives the liquid from the distal running catheter as an input to a distal region of the bag; and wherein the proximal running catheter receives the liquid from the bag as an output at a proximal region of the bag, the bag defining a flow path that connects the distal running catheter to the proximal running catheter and cools the surrounding tissue with the liquid.
